SEQUENCE LISTING (14 sequences)

SEQ ID NO: 1 - DNA, 1810 bp, Rattus rattus
  misc_feature (1705)..(1705): n is a, c, g, or t
gcggccgaggtacctgagacctcttctgctccagacgcgtccgcggccaggatgaagaggccgaagttaaagaaagcaagtaaacgtatgacctgtcataagcggtataaaatccagaaaaaggttcgagaacatcatcgaaaattaaggaaggaagctaaaaagcggggtcacaagaagcctaagaaggacccaggagttccaaatagtgctccctttaaagaggctcttcttcgtgaagctgagctaaggaaacagcagcttgaagaactaaaacagcagcagaaacttgacaggcaaaaagaacaagaacgaaaaagaaaacttgaaattagccctgatgatgagcaatctaatgtggaaactcaggaggaatctgatgagcccaaaataaagaaagctaaatcaggcaaacagaatccaaagaagttacattgtcaggaacttaaaaaggtgattgaagcctcagacattgtgttagaagttttggatgccagagatcctcttggttgcaggtgtcctcaagtagaagaagctgttatccaaagtggatgtaaaaaactagtacttgtattaaataagtcagatctagtaccaaaagagaatctagagaactggctaacttacttgaataaggaattgccaacagtggtgttcaaagcctcaacaaacttaaagaacagaaagaagacattcaagataaagaagaaagttgttccattccaaagtaaactctgctgtggcaaggaagcactgtggaagctccttggaggttttcagcagtcctgtggaaaaggagttcaggttggagtggttggtttcccaaatgtgggaaaaagcagcatcattaatagtttaaaacaagaaaggatttgcagtgttggagtttccatgggacttacaaggagtatgcagattgtccctttagacaaacagatcacaatcatagatagtccgtgcttcattatctcaccttgtaactcccctgctgcacttgccctccgaagtccagcaagtattgaagttctaagaccattggaggctgccagtgccatcctgtctcaggctgatagtcaacaggtggtgttaaaatatactgtcccggggtataaggattctctggatttttttactaaacttgctcagagaagaggtctgcaccaaaaaggtggaagcccaaatgtcgaaagtgctgctaagctgctatggtctgagtggacaggtgcctcattaggttactactgccatccccctgcatcctggaatcattctcctcattttaatgagaatattacagcaatcatgaagaggggctttaatctagaagaactagaaaagaataatgcacacagcatacaagtcctcaagggccctcatttaactaataaaatccttttccggtcttcgggcctgacaaatggaatactagaagaaaaggacatccccgaagagtcaccaaaacagacagaagaccaacaggatggtgatgatcaagaacatgttactggtgaaaaaaatgcagagatctcagatgtgactcctgtagaagagaccagggagatgtcacctgggcaatcaacagcaagtaaaccatctgacagatcctttatcttggataaaatgagtgaagaagacgatgcctatgactttaccacagattatatatagccttctaaatgttcaagtgtgctctgtacagtgtttntagattgctttggtatgatataaagtgtaaatcttgtgaatatgtatcatgttttaaattaaaaacaaaataaaaagtgtttgtataaaaaaaaaaaaaaaaaaa

SEQ ID NO: 2 - protein, 538 aa, Rattus rattus
MKRPKLKKASKRMTCHKRYKIQKKVREHHRKLRKEAKKRGHKKPKKDPGVPNSAPFKEALLREAELRKQQLEELKQQQKLDRQKEQERKRKLEISPDDEQSNVETQEESDEPKIKKAKSGKQNPKKLHCQELKKVIEASDIVLEVLDARDPLGCRCPQVEEAVIQSGCKKLVLVLNKSDLVPKENLENWLTYLNKELPTVVFKASTNLKNRKKTFKIKKKVVPFQSKLCCGKEALWKLLGGFQQSCGKGVQVGVVGFPNVGKSSIINSLKQERICSVGVSMGLTRSMQIVPLDKQITIIDSPCFIISPCNSPAALALRSPASIEVLRPLEAASAILSQADSQQVVLKYTVPGYKDSLDFFTKLAQRRGLHQKGGSPNVESAAKLLWSEWTGASLGYYCHPPASWNHSPHFNENITAIMKRGFNLEELEKNNAHSIQVLKGPHLTNKILFRSSGLTNGILEEKDIPEESPKQTEDQQDGDDQEHVTGEKNAEISDVTPVEETREMSPGQSTASKPSDRSFILDKMSEEDDAYDFTTDYI

SEQ ID NO: 3 - DNA, 1770 bp, Mus musculus
gaattcggcacgagggttgaaccgcagttccagttcgcacgtggcgcccgagaagtcgtggtgatcccgagacctcctctgctcctgaagcgtccgcggccaggatgaagaggcctaagttaaagaaagcgagtaaacgtatgacctgccataagcgatataaaattcaaaaaaaggtccgagaacatcatcgaaaattaaggaaggaagctaaaaaacggggtcacaagaagcctaggaaggacccaggtgttccaaatagtgctccctttaaagaggctcttcttcgtgaagctgaactaaggaaacagcagcttgaagaactaaaacagcagcagaaacttgataggcaaaaagagcaagaaaggaaaagaaaacttgaagttagccctggtgatgagcagtctaatgtggaaactagggaggaatctgacgagcccaaaagaaagaaagccaaagcaggcaaacagaatccaaagaagttacattgccaggaacttaaaaaggtgattgaagcctcagacattgtgttagaagttttggatgccagagatcctcttggttgcaggtgtcctcagatagaagaagctgttatccagagtgggagtaagaagctgatacttgtattaaataagtctgatctagtaccaaaggagaatttggagaactggctaaattatttgaataaagaattgccaaccgtggtgttcaaagcctcaacaaacttaaagaacagaaagacattcaagataaaaaagaagaaagttgttccattccaaagcaaaatctgctgtggcaaggaagccctttggaagcttcttggagattttcagcagtcctgtggaaaggatattcaagttggagtgattggtttcccaaatgtggggaaaagcagtgtcattaatagcttaaaacaagaatggatttgcaatgttgggatttccatgggacttacaaggagcatgcagattgtccctttagacaagcagatcacaatcatagacagtccatgcctaattatctcaccttgtaactcccccactgcacttgcccttcggagtccagcaagcattgaggaactaagaccgctggaggctgccagtgccattctgtctcaggctgataatgaacaggtggtgttaaaatatactgtccctgagtataaggattctctgcatttttttactaaacttgctcaaagaagaggtctgcaccaaaaaggtggaagcccaaatgtggaaagtgctgctaagctggtgtggtctgagtggacaggtgcctcattaggttactattgccatccccctgcatcctggaatcattctctgcattttaatgagaatattgcagcagtcatgaagaagggctttaatctagaagaactagaaaagaataatgcacacagcatacaagtcctcaagggccctcatttaactaatagaatcctttttcggtcttcgggcctgacaaatggaatactagacgagaaggacatagtcgaagagaccagggagctgtcacctgagcaatcaacagcaggtaagccatctgacgggtcgtctgccttggatagagcgagtcaagaggatgaaacctatgacttcaccacagattatatataaccgccacacactaacgtgctctctgtacgctgtgtagtttagtgtatgatataaactgtacatcttgtaaatatgtatcatgttataaattcaaaataaaatacaagtatttgcttgcaaaaaaaaaaaaaaaaaactcgactctaga

SEQ ID NO: 4 - protein, 538 aa, Mus musculus
MKRPKLKKASKRMTCHKRYKIQKKVREHHRKLRKEAKKRGHKKPRKDPGVPNSAPFKEALLREAELRKQQLEELKQQQKLDRQKEQERKRKLEVSPGDEQSNVETREESDEPKRKKAKAGKQNPKKLHCQELKKVIEASDIVLEVLDARDPLGCRCPQIEEAVIQSGSKKLILVLNKSDLVPKENLENWLNYLNKELPTVVFKASTNLKNRKTFKIKKKKVVPFQSKICCGKEALWKLLGDFQQSCGKDIQVGVIGFPNVGKSSVINSLKQEWICNVGISMGLTRSMQIVPLDKQITIIDSPCLIISPCNSPTALALRSPASIEELRPLEAASAILSQADNEQVVLKYTVPEYKDSLHFFTKLAQRRGLHQKGGSPNVESAAKLVWSEWTGASLGYYCHPPASWNHSLHFNENIAAVMKKGFNLEELEKNNAHSIQVLKGPHLTNRILFRSSGLTNGILDEKDIVEESPSQTEDQQDADDQENGSGERNAEISDVAPVEETRELSPEQSTAGKPSDGSSALDRASQEDETYDFTTDYI

SEQ ID NO: 5 - protein, 27 aa, Homo sapiens
LREAELRKQRLEELKQQQKLDRQKELE

SEQ ID NO: 6 - protein, 549 aa, Homo sapiens
MKRPKLKKASKRMTCHKRYKIQKKVREHHRKLRKEAKKQGHKKPRKDPGVPNSAPFKEALLREAELRKQRLEELKQQQKLDRQKELEKKRKLETNPDIKPSNVEPMEKEFGLCKTENKAKSGKQNSKKLYCQELKKVIEASDVVLEVLDARDPLGCRCPQVEEAIVQSGQKKLVLILNKSDLVPKENLESWLNYLKKELPTVVFRASTKPKDKGKITKRVKAKKNAAPFRSEVCFGKEGLWKLLGGFQETCSKAIRVGVIGFPNVGKSSIINSLKQEQMCNVGVSMGLTRSMQVVPLDKQITIIDSPSFIVSPLNSSSALALRSPASIEVVKPMEAASAILSQADARQVVLKYTVPGYRNSLEFFTMLAQRRGMHQKGGIPNVEGAAKLLWSEWTGASLAYYCHPPTSWTPPPYFNESIVVDMKSGFNLEELEKNNAQSIRAIKGPHLANSILFQSSGLTNGIIEEKDIHEELPKRKERKQEEREDDKDSDQETVDEEVDENSSGMFAAEETGEALSEETTAGEQSTRSFILDKIIEEDDAYDFSTDYV

SEQ ID NO: 7 - RNA, 23 nt, Artificial Sequence
  NS-specific siRNA
aagaacuaaaacagcagcagann

SEQ ID NO: 8 - RNA, 23 nt, Artificial Sequence
  Control siRNA for rat cells
aacauucagacugggaaauggnn

SEQ ID NO: 9 - RNA, 23 nt, Artificial Sequence
  Control siRNA for human cells
aaucagacguggaccagaagann

SEQ ID NO: 10 - protein, 549 aa, Artificial Sequence
  Amino acid sequence of a consensus nucleostemin
MKRPKLKKASKRMTCHKRYKIQKKVREHHRKLRKEAKKXGHKKPXKDPGVPNSAPFKEALLREAELRKQXLEELKQQQKLDRQKEXEXKRKLEXXPXXXXSNVEXXEXXXXXXXXXXKAKXGKQNXKKLXCQELKKVIEASDXVLEVLDARDPLGCRCPQXEEAXXQSGXKKLXLXLNKSDLVPKENLEXWLXYLXKELPTVVFXASTXXKXXXKXTXXXKXKXXXXPFXSXXCXGKEXLWKLLGXFQXXCXKXXXVGVXGFPNVGKSSXINSLKQEXXCXVGXSMGLTRSMQXVPLDKQITIIDSPXXIXSPXNSXXALALRSPASIEXXXPXEAASAILSQADXXQVVLKYTVPXYXXSLXFFTXLAQRRGXHQKGGXPNVEXAAKLXWSEWTGASLXYYCHPPXSWXXXXXFNEXIXXXMKXGFNLEELEKNNAXSIXXXKGPHLXNXILFXSSGLTNGIXXEKDIXEEXPXXXEXXQXXXXXXXXXDQEXXXXEXXXXXSXXXXXEETXEXXXXXXTAXXXSXXSXXLDXXXXEDXXYDFXTDYX

SEQ ID NO: 11 - DNA, 1926 bp, Artificial Sequence
  NS2
gctgcgcactcctggactggcgacgttgtgcttctaacagctctccgaggtccctgccggaagtgtaggaagaagcagacagatttgaacatctctgtttccagcttctctgatcatcatgatgaagattagacacaaaaacaaaaaaccaggtaaaggttccaaaggctgtaagaagcctgcaaggcaaaatgggaagaaagtaacctccagaccatcatctgctccccagattgttcatggcaatgaccatgccagtcgcgaggccgaattaaagaagaaaagggtcgaggagatgagggagaagcagcaagttgcccgggagcaagagagacagagacacaggaccatggagagctattgtcaggatgtcctgaaacgtcagcaggaatttgaacaaaaggaggaagttttgcaggaattaaacatgtttcctcagttggatgatgaggccacaaggaaggcctattacaaggaattccggaaggtggtagagtactctgatgtgattctggaagtcctagatgccagagacccattgggctgccgctgtttccagatggaggagactgtccttcgtgcagaaggcaacaagaagctggtcttggtcttaaataagatagatctcgttcccaaggagattgtggaaaagtggctggaataccttctcaatgaactgccaactgtggctttcaaggccagcacccagcatcatcaggtcaaaaacttgactcgttgtaaagttccagtggaccaggcctctgagtcgcttttgaaaagcagagcctgctttggagccgaaaatctcatgagggtcctggggaactattgtcgcctgggggaagtgcgtggccacattcgtgtgggtgttgtaggccttcccaatgtggggaagagcagtctgatcaatagcctgaagcgcagccgtgcttgtagtgtgggagctgttcctggtgtcacaaaattcatgcaggaggtctacctagacaagtttatcaggcttctggatgcaccaggcattgtcccaggacccaattcagaggtgggcaccatcctgcgtaattgcatccatgtgcagaagctggcagaccctgtgaccccggtggagaccatccttcagcgctgcaacctggaggagatttccagctactatggtgtatctggattccagacgactgagcactttctgactgcagtggcccatcgcttgggaaagaagaagaagggaggtgtatatagtcaggaacaggctgccaaagctgtgctggctgactgggtgagtgggaagatcagcttctatacactaccaccgcccactcacactctgcccacccatctcagtgctgagattgttaaggagatgactgaggtctttgatatagaagatactgagcacgccaatgaagacaccatggaatgcttagctgtgggagaatccgatgagctgttgggtgacatggacccacaagaaatggaggtcaggtggctccattctccactggtgaaaatagcagatgctattgaaaatagaagcaccgtgtataagattggaaatctcactgggtattgtaccaaaccaaaccgtaatcagatggggtggcctaaacgcaatgtggaccaccattgcccccaaaataaccgtgtagtagaggtcagttctgtggaccgccgcccgatgttgcagaggatcctggagacagacccacttcagcaaggccaggctctggaatctgccttgaagaataagaaaaaattgcagaagcgttcagataaaatcgccactaagttgtctgactccatgatgtccatgcttgacctctctggcaactccgatgactgtgcaggtgactgagcagctgaccttccccctcatactgcaagtactgcttcccgtgcgatgggagagtcagatgcctttcattctct

SEQ ID NO: 12 - protein, 576 aa, Artificial Sequence
  NS2
MKIRHKNKKPGKGSKGCKKPARQNGKKVTSRPSSAPQIVHGNDHASREAELKKKRVEEMREKQQVAREQERQRHRTMESYCQDVLKRQQEFEQKEEVLQELNMFPQLDDEATRKAYYKEFRKVVEYSDVILEVLDARDPLGCRCFQMEETVLRAEGNKKLVLVLNKIDLVPKEIVEKWLEYLLNELPTVAFKASTQHHQVKNLTRCKVPVDQASESLLKSRACFGAENLMRVLGNYCRLGEVRGHIRVGVVGLPNVGKSSLINSLKRSRACSVGAVPGVTKFMQEVYLDKFIRLLDAPGIVPGPNSEVGTILRNCIHVQKLADPVTPVETILQRCNLEEISSYYGVSGFQTTEHFLTAVAHRLGKKKKGGVYSQEQAAKAVLADWVSGKISFYTLPPPTHTLPTHLSAEIVKEMTEVFDIEDTEHANEDTMECLAVGESDELLGDMDPQEMEVRWLHSPLVKIADAIENRSTVYKIGNLTGYCTKPNRNQMGWPKRNVDHHCPQNNRVVEVSSVDRRPMLQRILETDPLQQGQALESALKNKKKLQKRSDKIATKLSDSMMSMLDLSGNSDDCAGD

SEQ ID NO: 13 - DNA, 2359 bp, Artificial Sequence
  NS3
gtcgacccacgcgtccgccaacgtgcttcacacaggacgctttcccggctgggaagtcgtgaagtggtttacctcgcgagtagcagagcggtgtcggtactgtcgtcaggatggtgaagcccaagtacaaaggacggagcaccatcaaccgctcggcggccagcaccaacccagatcgagtacagggagctggcggccaaaacatgagggatcggggcacaattcggcgcctgaatatgtacaggcaaaaggagcgcaggaacagtcgtggtaaagtgattaagccactgcagtatcagtcaactgtggcttctggcacagtggcccgagtggagccgaatattaaatggtttggaaatactcgtgtgatcaagcaggcatcattacaaaaatttcaagaggaaatggataaagttatgaaggatccatacaaagttgtcatgaaacaaagcaaattaccgatgtctcttctgcacgatcgaatccagcctcataacgcaaaagtccacattcttgatactgaaagctttgaaagtacatttggcccaaagtcacagagaaagcggccaaacttgtttgcaagtgatatgcaatcccttctagaaaacgctgaaatgtctactgagagctatgaccagggcaaggaccgtgatttggtgatggaggacactggtgtaagaaatgaagctcaagaagagatatataaaaaagggcagtcaaaaagaatatggggagaactctacaaggtgatagactcatcagatgttgtcgttcaagtccttgacgctagagatccgatgggcactcgttccccccacatcgaagcttacttgaaaaaggaaaaaccctggaaacatctcatttttgtactcaataagtgtgaccttgttccaacttgggcaaccaaacgatgggttgctgtgctctcccaggactacccaacactggctttccatgcgagcctcaccaatccctttgggaagggagcattcattcagcttctgcggcagtttgggaagttgcacacagacaagaaacaaatcagtgttgggttcattggctatccaaatgtaggcaagagctctgtgattaatacattacgatccaagaaagtttgcaacgtggcccccattgctggagaaacaaaggtctggcagtatattaccttgatgcgtcgtatattcctgattgactgccctggtgtggtttacccatctgaggactcagagaccgacattgtgctcaaaggagtggttcaagttgagaaaattaaagctcctcaagaccacattggtgctgtccttgaacgagcaaagccagagtatatcagcaagacgtacaagattgagtcctgggagaacgcggaggactttcttgagaagctagctctccgcactgggaagttactgaagggtggagagcctgacatgctgactgtgagcaagatggttctcaatgactggcagagaggccgaatccctttctttgtcaagccgcccaatgcagagctaccgaccgattcccagcttccaccatcctcaccattggaagttcccacagaaacaacccagaacaacccagaagaagagaccacagaaacagaagttgaaaggtcagactctatcactgaaaaggagccagaaggagactgttctcaggatagaaactcagagatgcaacagatcctcgcacgagttcgccagaactttggcaaaatcaacgtggggcctcagttttctgcggatgacctggtgcctgtggagatgtcagacttggaagatctggaaagctctggggaagaggaagaacaggagcaggaacagccaggggaggatgccgaggaagagcgctccccagacactcaggaggaaccagtgggaaacgacaccaaggccgtgctcagagccctggatgagaagattgccaagtaccagaggtttctaaataaagctaaagctaaaaagttctctgccgtcagaatatccaaggacttaagtgaaaaggtttttgcaaaatacaaagaagagaagaaaacatctgcagaagacagtgatgcagcacccaccaaaaaggcaaggaagtgggatgcacagatggaagaagaaccttcaaataagactcagaggatgctgacgtgtaaggaacggaggagagcagcacggcagcaacaatccaaaaaagttggtgtgcgttactacgagacacacaatgtgaaaaacaggaacaggaacaaaaagaagacgagcgactcagagggacagaaacacagacgcaacaagttcagacagaagcagtaactgcgagaaagctgtttattaaattatacaaaaataaaaaaaaaaaaaaaaagggcggccgc

SEQ ID NO: 14 - protein, 728 aa, Artificial Sequence
  NS3
MVKPKYKGRSTINRSAASTNPDRVQGAGGQNMRDRGTIRRLNMYRQKERRNSRGKVIKPLQYQSTVASGTVARVEPNIKWFGNTRVIKQASLQKFQEEMDKVMKDPYKVVMKQSKLPMSLLHDRIQPHNAKVHILDTESFESTFGPKSQRKRPNLFASDMQSLLENAEMSTESYDQGKDRDLVMEDTGVRNEAQEEIYKKGQSKRIWGELYKVIDSSDVVVQVLDARDPMGTRSPHIEAYLKKEKPWKHLIFVLNKCDLVPTWATKRWVAVLSQDYPTLAFHASLTNPFGKGAFIQLLRQFGKLHTDKKQISVGFIGYPNVGKSSVINTLRSKKVCNVAPIAGETKVWQYITLMRRIFLIDCPGVVYPSEDSETDIVLKGVVQVEKIKAPQDHIGAVLERAKPEYISKTYKIESWENAEDFLEKLALRTGKLLKGGEPDMLTVSKMVLNDWQRGRIPFFVKPPNAELPTDSQLPPSSPLEVPTETTQNNPEEETTETEVERSDSITEKEPEGDCSQDRNSEMQQILARVRQNFGKINVGPQFSADDLVPVEMSDLEDLESSGEEEEQEQEQPGEDAEEERSPDTQEEPVGNDTKAVLRALDEKIAKYQRFLNKAKAKKFSAVRISKDLSEKVFAKYKEEKKTSAEDSDAAPTKKARKWDAQMEEEPSNKTQRMLTCKERRRAARQQQSKKVGVRYYETHNVKNRNRNKKKTSDSEGQKHRRNKFRQKQ

(19) United States
(12) Patent Application Publication
(10) Pub. No.: US 2006/0009379 A1
(43) Pub. Date: Jan. 12, 2006

(54) Methods for controlling proliferation of cells

(75) Inventors: Robert Y.L. Tsai, SILVER SPRING, MD (US); Ronald D.G. McKay, Bethesda, MD (US)

Correspondence Address: KLARQUIST SPARKMAN, LLP, 121 S.W. SALMON STREET, SUITE #1600, ONE WORLD TRADE CENTER, PORTLAND, OR, 97204-2988, US

(73) Assignee: THE GOVERNMENT OF THE UNITED STATES OF AMERICAN AS REPRESENTED BY THE DEPT. OF HEALTH AND HUMAN SVC, ROCKVILLE, MD (US)

(21) Appl. No.: 10/530,340

(22) PCT Filed: Oct. 1, 2003
(86) PCT No.: PCT/US03/31321

§ 371 Date: Apr. 1, 2005

Related U.S. Application Data
(60) Provisional application No. 60/415,867, filed on Oct. 2, 2002.
(60) Provisional application No. 60/442,005, filed on Jan. 22, 2003.

(51) Int. Cl. A61K 38/17

(52) U.S. Cl. 514/12

(57) ABSTRACT

An isolated nucleostemin polypeptide is disclosed herein. The nucleostemin polypeptide includes an amino acid sequence at least 85% identical to SEQ ID NO: 2. In several examples, the polypeptide regulates cell differentiation, cell proliferation, or both. Nucleic acids encoding these polypeptides, vectors including the nucleic acids, and host cells transfected with these nucleic acids are also disclosed. Methods for inducing differentiation, inhibiting proliferation, and inducing senescence of a cell by altering the level of a nucleostemin polypeptide including an amino acid sequence at least 80% identical to SEQ ID NO: 2 are also disclosed. Methods for screening for agents that affect proliferation, differentiation, or senescence of cells are also disclosed.

### PRIORITY CLAIM

This application claims the benefit of U.S. Provisional Application No. 60/415,867, filed Oct. 2, 2003, and U.S. Provisional Application No. 60/442,005, filed Jan. 22, 2003. Both of these provisional applications are incorporated by reference in their entirety.

### FIELD

This application relates to the field of cell cycle control, specifically to the methods of inducing differentiation and inhibiting proliferation of cells by altering nucleostemin activity.

### BACKGROUND

Stem cells have been identified in several somatic tissues including the nervous system, bone marrow, epidermis, skeletal muscle, and liver. This ‘set-aside’ population of cells is believed to be responsible for maintaining homeostasis within individual tissues in adult animals. The number of stem cells and their decision to differentiate must be tightly controlled during embryonic development and in the adult animal to avoid premature aging or tumor formation. Different somatic stem cells share the properties of self-renewal and multi-developmental potential, suggesting the presence of common cellular machinery.

Embryonic stem (ES) cells can proliferate indefinitely in an undifferentiated state. Furthermore, ES cells are totipotent cells, meaning that they can generate all of the cells present in the body (bone, muscle, brain cells, etc.). ES cells have been isolated from the inner cell mass of the developing murine blastocyst (Evans et al., Nature 292:154-156, 1981; Martin et al., Proc. Natl. Acad. Sci. U.S.A. 78:7634-7636, 1981; Robertson et al., Nature 323:445-448, 1986; Doetschman et al., Nature 330:576-578, 1987; and Thomas et al., Cell 51:503-512, 1987; U.S. Pat. No. 5,670,372). Additionally, human cells with ES properties have recently been isolated from the inner blastocyst cell mass (Thomson et al., Science 282:1145-1147, 1998) and developing germ cells (Shamblott et al., Proc. Natl. Acad. Sci. U.S.A. 95:13726-13731, 1998) (see also U.S. Pat. No. 6,090,622, WO 00/70021 and WO 00/27995).

Cancer is an abnormal state in which uncontrolled proliferation of one or more cell populations interferes with normal biological functioning. The proliferative changes are usually accompanied by other changes in cellular properties, including reversion to a less differentiated, more developmentally primitive state. The in vitro correlate of cancer is called cellular transformation. Transformed cells generally display several or all of the following properties: spherical morphology, expression of fetal antigens, growth-factor independence, lack of contact inhibition, anchorage-independence, and growth to high density.

There is growing interest in the analysis of patterns of gene expression in cells, such as cancer cells and stem cells, using microarray technology. However, few studies have identified an individual gene product that functions in the complex network of signals in developing tissues to promote differentiation and decrease proliferation. Disclosed herein is the nucleostemin polypeptide and polynucleotides encoding nucleostemin, which are of use in promoting differentiation and decreasing proliferation of cells, such as stem cells, including embryonic stem cells and tumor cells.

### SUMMARY

It has now been discovered that cell differentiation and proliferation can be controlled by a newly discovered polypeptide, referred to herein as nucleostemin. In one embodiment, an isolated nucleostemin polypeptide includes an amino acid sequence at least 85% identical to SEQ ID NO: 2. In several examples, the polypeptide regulates cell differentiation, cell proliferation, or both. In one specific, non-limiting example, the polypeptide has a sequence as set forth as SEQ ID NO: 2. In another specific, non-limiting example, the polypeptide has a sequence set forth as SEQ ID NO: 4. Polynucleotides are disclosed that encode a nucleostemin polypeptide including an amino acid sequence at least 85% identical to SEQ ID NO: 2. Vectors including these polynucleotides, and host cells transfected with these polynucleotides, are also disclosed.

A method for inducing differentiation of a cell is also disclosed herein. The method includes altering the level of a nucleostemin polypeptide, including an amino acid sequence at least 80% identical to SEQ ID NO: 6, thereby inducing differentiation of the cell.

A method for inhibiting proliferation of a cell is also disclosed herein. The method includes altering the level of a nucleostemin polypeptide including an amino acid sequence at least 80% identical to SEQ ID NO: 6, thereby inhibiting proliferation of the cell.

A method for inducing senescence of a cell is also disclosed herein. The method includes altering the level of a nucleostemin polypeptide, including an amino acid sequence at least 80% identical to SEQ ID NO: 6, thereby inducing senescence of the cell.

A method is disclosed for screening for agents that affect differentiation or proliferation of a cell. The method includes contacting p53 and a nucleostemin polypeptide comprising an amino acid sequence at least 80% identical to SEQ ID NO: 6 with an agent of interest in vitro and evaluating binding of p53 and the nucleostemin polypeptide. A decrease in the binding of p53 and the nucleostemin polypeptide as compared to a control indicates that the agent affects differentiation or proliferation of the cell.

A method is disclosed for decreasing proliferation of a tumor cell in a subject. The method includes administering to the subject a therapeutically effective amount of an agent that alters the level of a nucleostemin polypeptide, wherein the polypeptide includes an amino acid sequence at least 80% identical to SEQ ID NO: 6.

The foregoing and other features and advantages will become more apparent from the following detailed description of several embodiments, which proceeds with reference to the accompanying figures.

### BRIEF DESCRIPTION OF THE FIGURES

FIGS. 1A-1D are a set of digital images and diagrams on the cloning of nucleostemin. FIG. 1A is a set of digital images of rat embryonic cortical stem cells (Stein). Stem were used as the tester and 8 day serum-differentiated cultures (S8) as the driver in a cDNA subtractive screen. Left: nestin and BrdU staining; middle: Tuj1 for neurons; GFAP for astrocytes, right: O4 for oligodendrocytes, green. Scale bar: 25 μm (stem); 50 μm (S8). FIG. 1B is a digital image Northern analysis of subtractive outputs and controls. Sc, stem cells; F2 and F8, 2d and 8d differentiation in 10% serum and bFGF (20 ng/ml); S2 and S8, 2 day and 8 day differentiation in 10% serum without bFGF; NS, nucleostemin; HMGCoR, 3-hydroxyl, 3-methyl glutaryl CoA reductase; Tβ-3, neuron-specific type III α-tubulin; GFAP, glial fibrillary acidic protein; MBP, myelin basic protein. FIG. 1C is a schematic diagram of nucleostemin protein. CC, coiled-coil domain; G4 and G1, GTP-binding motifs; B and A, basic and acidic domains; filled boxes, NLS. FIG. 1D is a sequence alignment of rat (SEQ ID NO: 2), mouse (SEQ ID NO: 4) and human nucleostemin (SEQ ID NO: 6). Shaded areas correspond to the structural domains indicated in FIG. 1C. Underline, NLS; bases are shaded so that lighter amino acids are conserved residues in all species; darker amino acids are conserved residues in two of the three species.

FIGS. 2A-2P are a set of digital images showing protein expression and localization of nucleostemin. FIG. 2A is a Western blot with α-nucleostemin (2438) antiserum identified a major band in undifferentiated stem cells (stem), but not in 2 day and 7 day serrum-differentiated cultures (SeD2, SeD7). α-Tub: anti-α-tubulin antibody used as internal loading control. Immunodetection of endogenous nucleostemin in rat embryonic cortical stem cells (FIGS. 2B, 2D), neurosphere cells derived from adult SVZ (FIG. 2F), ES cells (FIG. 2G), and corresponding DAPI counter stain (FIGS. 2C, 2E, 2H, inset in 2F). CHO cells transfected with a nucleostemin-GFP fusion construct revealed strong nucleolar fluorescence (FIGS. 2I, 2J). Nucleostemin is co-localized with nucleolin immunoreactivity in HEK293 cells (FIGS. 2K, 2L). Immunostaining of nucleostemin in three human cancer cell lines: H1299 (FIG. 2M), U20S (FIG. 2N), and Saos-2 (FIG. 20). FIG. 2P shows the distribution of nucleostemin during cell cycle. CHO cells at prophase (Pro), metaphase (Meta), anaphase (Ana) and telophase (Tel) were identified by DAPI staining. Scale bar: 50 μm for FIGS. 2G, 2H, 25 μm for FIG. 2F, and 10 μm for others.

FIGS. 3A-3D are a set of digital images showing the in vivo distribution of nucleostemin. FIG. 3A is a digital image of a developmental Western blot of nucleostemin protein expressed in mouse forebrains. The same blot was reprobed with different antibodies. NS, nucleostemin; E8.5, embryonic day 8.5; P10, postnatal day 10; Ad, adult; PCNA, proliferating cell nuclear antigen; α-Tub, α-tubulin. FIG. 3B is a digital image of a multi-tissue Northern blot on adult rat. Ctx, cortex; Cbm, cerebellum; Ht, heart; Lg, lung; Sk, skeletal muscle; Te, testis; Lv, liver; Int, intestine; Kd, kidney; Sp, spleen. FIGS. 3C and 3D are digital images of the regional distribution of nucleostemin in transverse sections of the forebrain (FIG. 3C) and spinal cord (FIG. 3D) of E10.5 mouse embryos by immunohistochemistry (1643). Nucleostemin signals appear as brown dots (indicated by arrows) and are seen in the neuroepithelium (ne) and mesenchyme (mes). The signal is stronger in the forebrain than in the spinal cord. Within the spinal cord, the ventricular zone (vz) expressed higher levels of nucleostemin than the mantle zone (mz). Lower panel in FIG. 3C represents a high power view of the upper panel. Abbreviations: v, ventricle. Scale bar: 50 μm for FIG. 3C top panel and FIG. 3D; 25 μm for FIG. 3C lower panel.

FIGS. 4A-4E are a set of images showing nucleostemin protein is down-regulated in both dividing and non-dividing progeny during differentiation.

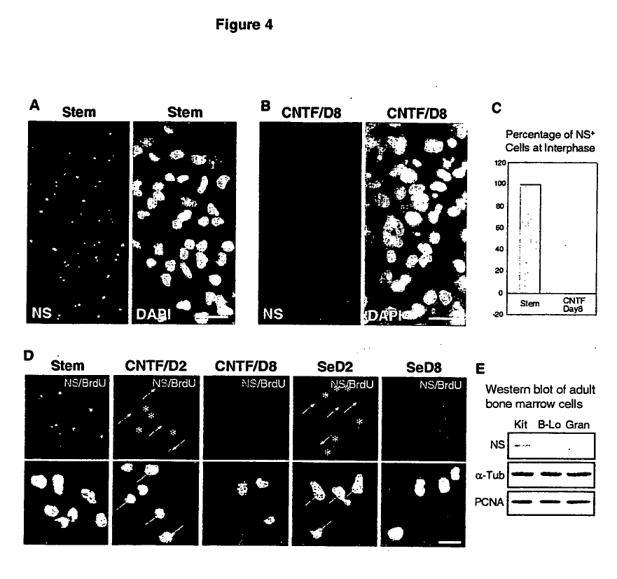

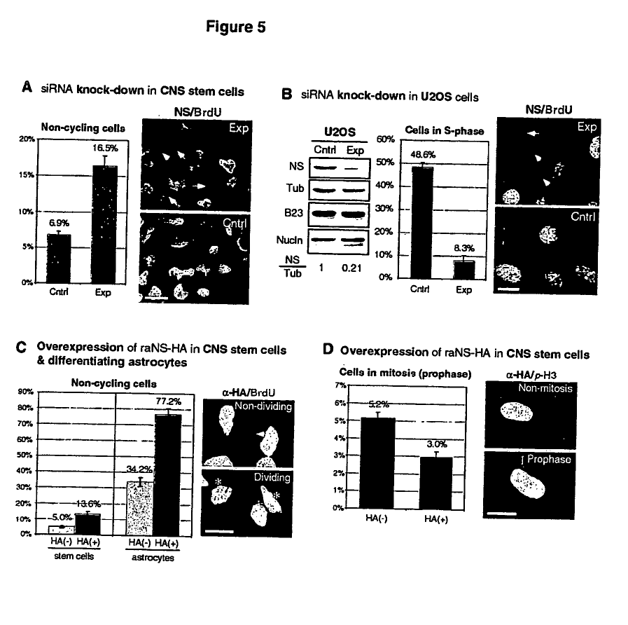

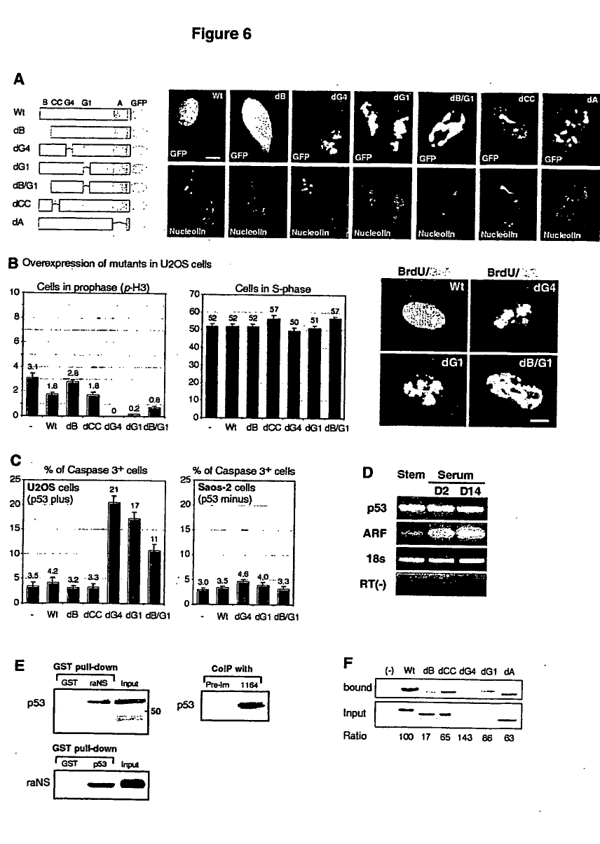

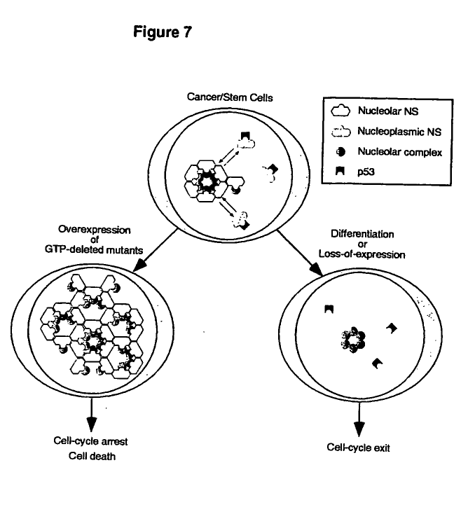

Nucleostemin is expressed in all the rat cortical stem cells (FIG. 4A, Stem) but not in the 8d CNT-differentiated culture (FIG. 4B, CNTF/D8). FIG. 4C is a bar graph showing the percentage of nucleostemin⁺ cells in FIGS. 4A and 4B. FIG. 4D is a digital image of nucleostemin (NS)-BrdU double-labeled immunofluorescence in undifferentiated stem cells (Stem), 2d and 8d differentiated cultures with CNTF (10 ng/ml, CNTF/D2 and CNTF/D8) or 10% FBS (SeD2 and SeD8). All cultures are pulse-labeled with 15 minutes of BrdU (40 μM) before fixation. Arrow and asterisk indicates BrdU-labeled and non-labeled cells. Both species have low level of nucleostemin expression in the differentiated cultures. Scale bar: 20 μm. FIG. 4E is a digital image of a Western analysis of lineage-depleted, c-kit⁺ bone marrow cells (Kit), B-lymphocytes (B-Lo), and granulocytes (Gran) from adult mice.

FIGS. 5A-5D are a set of images and graphs showing perturbed expression of nucleostemin drives cells out of the cell cycle.

FIG. 5A is a digital image and graph showing siRNA knockdown of nucleostemin in cortical stem cells. (Left panel) The percentage of non-cycling cells increases in cultures transfected with NS-specific siRNA (Exp) as compared with cultures treated with control siRNA (Cntrl). (Right panel) Nucleostemin (NS)-BrdU double-labeled Immunofluorescence of culture treated with NS-specific (Exp) or control (Cntrl) siRNA. Arrows indicate non-dividing cells. Scale bar: 10 μm.

FIG. 5B is a digital image and graph of siRNA knockdown of nucleostemin in U2OS cells. (Left panel) Western analysis of the expression level of nucleostemin (NS), α-tubulin (α-Tub), B23, and nucleolin (Nucln). The amounts of nucleostemin protein (2 days after transfection) were standardized to the amounts of α-tubulin on the same blot and expressed as ratios to the control (middle panel). The percentage of cells in S-phase is lower in cultures transfected with NS-specific siRNA (Exp) than the control (Cntrl). (Right panel) NS-BrdU double-labeled immunofluorescence of control and experimental cultures. Arrows indicate cells without nucleostemin staining. Scale bar: 20 μm.

FIG. 5C is a graph and digital image showing overexpression of HA-tagged nucleostemin in bFGF-maintained, undifferentiated culture (stem cells) or CNTF-induced, 2d differentiated culture (astrocytes). The percentage of non-dividing cells among raNS-expressing cells (HA(+), left panel) is higher than the control-transfected cells (HA(−), left panel) in both the bFGF and the 2 day CNTF cultures. (Right panel) Arrow indicates non-dividing, raNS-expressing cells and asterisk indicates dividing, raNS-expressing cells. Scale bar: 10 μm.

FIG. 5D is a graph and digital image showing overexpression of HA-tagged nucleostemin (HA(+), left panel; darker staining, right panel) in undifferentiated stem cell culture decreases the percentage of cells in prophase, revealed by α-p-H3 staining (bright staining, right panel). Scale bar: 10 μm. In all the studies in this figure, statistical analyses were carried out by scoring randomly chosen HPFs (high power fields) from two independent triplicate experiments (n=6); error bars represent standard error mean (s.e.m.).

FIGS. 6A-6F are schematic diagrams, digital images, and graphs showing the deletion analysis and regulatory control of nucleostemin

FIG. 6A is a schematic diagram (left panel) and digital images showing the intracellular distribution (right panels) of mutant nucleostemins lacking individual structural domains. Scale bar. 10 μm.

FIG. 6B is a set of graphs and digital images showing overexpressing mutant nucleostemin decreases the number of cells in prophase (left panel, revealed by p-H3 staining), particularly with the dG4 and dG1 mutants, but not the number of cells in S-phase (middle panel) in U2OS cells. Statistical analyses were carried out by scoring randomly chosen HPFs from two independent triplicate experiments (n=6); error bars represent s.e.m. (Right panel) BrdU-GFP immunofluorescence shows the patterns of BrdU labeling in wild-type or mutants-transfected cells during S-phase. Scale bar: 10 μm.

FIG. 6C is a set of graphs showing overexpressing dG4, dG1, and, to a lesser extent, dB/G1 mutant nucleostemin increases the number of apoptotic cells in U2OS cells (left panel), but not in Saos-2 cells (right panel). Statistical analyses were carried out by scoring randomly chosen HPFs from two independent triplicate experiments (n=6). Error bars: s.e.m.

FIG. 6D is a digital image showing the expression levels of p53 and ARF in CNS stem cells and cultures treated with 10% serum for 2d and 14d were measured by semi-quantitative RTPCR. 18S rRNA with 2:8 primer to Competimer ratio was used as a positive control and RT(−) reaction, carried out without RT in the 1^{st} strand synthesis reaction, was used as a negative control. Amplication cycles are: p53 (29×), ARF (35×), 18s (23×), RT(−) (33×).

FIG. 6E is a set of digital images showing that nucleostemin and p53 exist in a protein complex. In GST pull-down assays, p53 is specifically retained in the GST-raNS fraction and later detected by α-nucleostemin (2438) Western blot analysis (left upper panel) and vice versa (left lower panel). Such interaction can also be shown by coimmunoprecipitation of endogenous p53 and nucleostemin (right panel) in HEK293 cell lysate by α-nucleostemin (1164) antiserum but not by preimmune serum (Pre-Im).

FIG. 6F is a digital image of mapping of p53-interacting region in nucleostemin by GST pull-down assay. Same amount of GST-p53 and comparable amount of HA-tagged nucleostemin proteins (with the exception of dG4 and dG1) was used in each reaction. The retained proteins (bound) were detected by α-HA Western blot and expressed as ratios to the input.

FIG. 7 is a schematic diagram illustrating a model of the GTP-regulated control of nucleostemin on stem cell proliferation. During normal cell proliferation in stem cells or cancer cells, nucleostemin is distributed predominantly in the nucleolus (represented by white polygonal symbols) and less in the nucleoplasm (represented by dark gray polygonal symbols). The association of nucleostemin with the nucleolar complex (represented by gray circles) is mediated through the N-terminal basic domain of nucleostemin. The downregulation of nucleostemin, during stem cell differentiation or in knockdown experiments, leads to cell-cycle exit. Overexpression of mutants lacking GTP-binding motifs causes nucleolar aggregates, which leads to cell-cycle arrest in the late S phase and cell death, suggesting the GTP-binding domain plays a role in the dynamic shutting of nucleostemin between in the nucleolar and nucleoplasmic compartments and is important for its normal function. The interaction of nucleostemin with p53 is mediated through its N-terminal basic domain and presumably takes place in the nucleoplasm.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

SEQ ID NO: 1 is the nucleic acid sequence of rat nucleostemin.

SEQ ID NO: 2 is the amino acid sequence of rat nucleostemin.

SEQ ID NO: 3 is the nucleic acid sequence of mouse nucleostemin.

SEQ ID NO: 4 is the amino acid sequence of mouse nucleostemin.

SEQ ID NO: 5 is the nucleic acid sequence of human nucleostemin.

SEQ ID NO: 6 is the amino acid sequence of human nucleostemin.

SEQ ID NO: 7 is the NS-specific siRNA.

SEQ ID NO: 8 is the control siRNA for rat cells.

SEQ ID NO: 9 is the control siRNA for human cells.

SEQ ID NO: 10 is the amino acid sequence of a consensus nucleostemin.

### DETAILED DESCRIPTION

I. Abbreviations

Ana: anaphase

BrdU: bromodeoxyuridine

CC: coil-coiled

CHO: chines hamster ovary cells

CNS: central nervous system

CNTF: ciiary neurotrophic factor

CNTF: ciliary neurotrophic factor

DAPI: 4′,6-Diamidino-2-phenylindole

dA: acidic domain

dB: basic domain

E: embryonic day, as in E10

ES: embyronic stem cells

GFAP: glial fibrillary acidic protein;

GFP: green fluorescent protein

HA: hemoagglutinin

HMGCoR: 3-hydroxyl, 3-methyl glutaryl CoA reductase

MBP: myelin basic protein

Met: metaphase

muNS: mouse nucleostemin

NS: nucleostemin

PCR: polymerase chain reaction

raNS: ratnucleostemin

RB: retinoblastoma

SiRNA: small inhibitory RNA

Tβ-3: neuron-specific type III α-tubulin

Tel: telophase

II. Terms

Unless otherwise noted, technical terms are used according to conventional usage. Definitions of common terms in molecular biology may be found in Benjamin Lewin, Genes V, published by Oxford University Press, 1994 (ISBN 0-19-854287-9); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8).

In order to facilitate review of the various embodiments of this disclosure, the following explanations of specific terms are provided:

Agent: Any polypeptide, compound, small molecule, organic compound, salt, polynucleotide, or other molecule of interest

Alter: A change in an effective amount of a substance of interest, such as a polynucleotide or polypeptide. The amount of the substance can changed by a difference in the amount of the substance produced, by a difference in the amount of the substance that has a desired function, or by a difference in the activation of the substance. The change can be an increase or a decrease. The alteration can be in vivo or it vitro. In several embodiments, altering an effective amount of a polypeptide or polynucleotide is at least about a 50%, 60%, 70%, 80%, 90%, 95%, 96%, 97%, 98%, 99%, or 100% increase or decrease in the effective amount (level) of a substance. In another embodiment, an alteration in polypeptide or polynucleotide affects a physiological property of a cell, such as the differentiation, proliferation, or senescence of the cell.

Animal: Living multi-cellular vertebrate organisms, a category that includes, for example, mammals and birds. The term mammal includes both human and non-human mammals. Similarly, the term “subject” includes both human and veterinary subjects.

Antibiotic Resistance Cassette: A nucleic acid sequence encoding a selectable marker which confers resistance to that antibiotic in a host cell in which the nucleic acid is translated. Examples of antibiotic resistance cassettes include, but are not limited to: kanamycin, ampicillin, tetracycline, chloramphenicol, neomycin, hygromycin, and zeocin.

Antisense, Sense, and Antigene: DNA has two strands, a 5′→3′ strand, referred to as the plus strand, and a 3′→5′ strand, referred to as the minus strand. Because RNA polymerase adds nucleic acids in a 5′→3′ direction, the minus strand of the DNA serves as the template for the RNA during transcription. Thus, the RNA formed will have a sequence complementary to the minus strand, and identical to the plus strand (except that U is substituted for T).

Antisense molecules are molecules that are specifically hybridizable or specifically complementary to either RNA or the plus strand of DNA. Sense molecules are molecules that are specifically hybridizable or specifically complementary to the minus strand of DNA. Antigene molecules are either antisense or sense molecules directed to a DNA target. An antisense RNA (asRNA) is a molecule of RNA complementary to a sense (encoding) nucleic acid molecule.

cDNA (complementary DNA): A piece of DNA lacking internal, non-coding segments (introns) and regulatory sequences that determine transcription. cDNA is synthesized in the laboratory by reverse transcription from messenger RNA extracted from cells.

Degenerate variant: A polynucleotide encoding a nucleostemin that includes a sequence that is degenerate as a result of the genetic code. There are 20 natural amino acids, most of which are specified by more than one codon. Therefore, all degenerate nucleotide sequences are included as long as the amino acid sequence of the nucleosternin polypeptide encoded by the nucleotide sequence is unchanged.

Differentiation: Refers to the process whereby relatively unspecialized cells (e.g., embryonic cells) acquire specialized structural and/or functional features characteristic of mature cells. Similarly, “differentiate” refers to this process. Typically, during differentiation, cellular structure alters and tissue-specific proteins appear.

Effective amount or Therapeutically effective amount: The amount of agent sufficient to prevent, treat, reduce and/or ameliorate the symptoms and/or underlying causes of any of a disorder or disease. In one embodiment, an “effective amount” is sufficient to reduce or eliminate a symptom of a disease. In another embodiment, an effective amount is an amount sufficient to overcome the disease itself.

Embryonic stem (ES) cells: Pluripotent cells isolated from the inner cell mass of the developing blastocyst. “ES cells” can be derived from any organism. ES cells can be derived from mammals. In one embodiment, ES cells are produced from mice, rats, rabbits, guinea pigs, goats, pigs, cows and humans. Human and murine derived ES cells are preferred. ES cells are totipotent cells, meaning that they can generate all of the cells present in the body (bone, muscle, brain cells, etc.). Methods for producing murine ES cells can be found in U.S. Pat. No. 5,670,372, herein incorporated by reference. Methods for producing human ES cells can be found in U.S. Pat. No. 6,090,622, WO 00/70021 and WO 00/27995, herein incorporated by reference.

Enhancer: A cis-regulatory sequence that can elevate levels of transcription of a coding sequence from an adjacent promoter. Many tissue specific enhancers can determine spatial patterns of gene expression in higher eukaryotes. Enhancers can act on promoters over many tens of kilobases of DNA and can be 5′ or 3′ to the promoter they regulate. Enhancers can function either by initiating transcription from a promoter operably linked to the enhancer or by providing binding sites for gene regulatory proteins that increase transcription of a minimal promoter.

Epitope: An antigenic determinant. These are particular chemical groups or peptide sequences on a molecule that are antigenic, i.e. that elicit a specific immune response. An antibody specifically binds a particular antigenic epitope on a polypeptide.

Expand: A process by which the number or amount of cells in a cell culture is increased due to cell division. Similarly, the terms “expansion” or “expanded” refers to this process. The terms “proliferate,” “proliferation” or “proliferated” may be used interchangeably with the words “expand,” “expansion”, or “expanded.” Typically, during an expansion phase, the cells do not differentiate to form mature cells.

Heterologous: A heterologous sequence is a sequence that is not normally (i.e. in the wild-type sequence) found adjacent to a second sequence. In one embodiment, the sequence is from a different genetic source, such as a virus or organism, than the second sequence.

Host cells: Cells in which a vector can be propagated and its DNA expressed. The cell may be prokaryotic or eukaryotic. The term also includes any progeny of the subject host cell. It is understood that all progeny may not be identical to the parental cell since there may be mutations that occur during replication. However, such progeny are included when the term “host cell” is used.

Hybridization: The process wherein oligonucleotides and their analogs bind by hydrogen bonding, which includes Watson-Crick, Hoogsteen or reversed Hoogsteen hydrogen bonding, between complementary bases. Generally, nucleic acid consists of nitrogenous bases that are either pyrimidines (Cytosine (C), uracil (U), and thymine(T) or purines (adenine (A) and guanine (G)). These nitrogenous bases form hydrogen bonds consisting of a pyrimidine bonded to a purine, and the bonding of the pyrimidine to the purine is referred to as “base pairing.” More specifically, A will bond to T or U, and G will bond to C. “Complementary” refers to the base pairing that occurs between two distinct nucleic acid sequences or two distinct regions of the same nucleic acid sequence.

“Specifically hybridizable” and “specifically complementary” are terms which indicate a sufficient degree of complementarity such that stable and specific binding occurs between the oligonucleotide (or its analog) and the DNA or RNA target. The oligonucleotide or oligonucleotide analog need not be 100% complementary to its target sequence to be specifically hybridizable. An oligonucleotide or analog is specifically hybridizable when binding of the oligonucleotide or analog to the target DNA or RNA molecule interferes with the normal function of the target DNA or RNA, and there is a sufficient degree of complementarity to avoid non-specific binding of the oligonucleotide or analog to non-target sequences under conditions in which specific binding is desired, for example, under physiological conditions in the case of in vivo assays. Such binding is referred to as “specific hybridization.”

Hybridization conditions resulting in particular degrees of stringency will vary depending upon the nature of the hybridization method of choice and the composition and length of the hybridizing nucleic acid sequences. Generally, the temperature of hybridization and the ionic strength (especially the Na⁺ concentration) of the hybridization buffer will determine the stringency of hybridization.

Nucleic acid duplex or hybrid stability is expressed as the melting temperature or Tm, which is the temperature at which a probe dissociates from a target DNA This melting temperature is used to define the required stringency conditions. If sequences are to be identified that are related and substantially identical to the probe, rather than identical, then it is useful to first establish the lowest temperature at which only homologous hybridization occurs with a particular concentration of salt (e.g., SSC or SSPE). Then, assuming that 1% mismatching results in a 1° C. decrease in the Tm, the temperature of the final wash in the hybridization reaction is reduced accordingly (for example, if sequences having >95% identity with the probe are sought, the final wash temperature is decreased by 5° C.). In practice, the change in Tm can be between 0.5° C. and 1.5° C. per 1% mismatch. The parameters of salt concentration and temperature can be varied to achieve the optimal level of identity between the probe and the target nucleic acid. Calculations regarding hybridization conditions required for attaining particular degrees of stringency are discussed by Sambrook et al. (ed.), Molecular Cloning: A Laboratory Manual, 2nd ed., vol. 1-3, Cold Spring Harbor Laboratory Press, Cold Spring Harbor, N.Y., 1989, chapters 9 and 11, herein incorporated by reference.

For purposes of this disclosure, “stringent conditions” encompass conditions under which hybridization will only occur if there is less than 30% mismatch between the hybridization molecule and the target sequence. “Stringent conditions” may be broken down into particular levels of stringency for more precise definition. Thus, as used herein, “moderate stringency” conditions are those under which molecules with more than 30% sequence mismatch will not hybridize; conditions of “medium stringency” are those under which molecules with more than 20% mismatch will not hybridize, and conditions of “high stringency” are those under which sequences with more than 10% mismatch will not hybridize.

Molecules with complementary nucleic acids form a stable duplex or triplex when the strands bind, or hybridize, to each other by forming Watson-Crick, Hoogsteen or reverse Hoogsteen base pairs. Stable binding occurs when an oligonucleotide remains detectably bound to a target nucleic acid sequence under the required conditions. “Complementarity” is the degree to which bases in one nucleic acid strand base pair with the bases in a second nucleic acid strand. Complementarity is conveniently described by the percentage, i.e. the proportion of nucleotides that form base pairs between two strands or within a specific region or domain of two strands. For example, if 10 nucleotides of a 15 nucleotide oligonucleotide form base pairs with a targeted region of a DNA molecule, that oligonucleotide is said to have 66.67% complementarity to the region of DNA targeted.

In the present disclosure, “sufficient complementarity” means that a sufficient number of base pairs exist between the oligonucleotide and the target sequence to achieve detectable binding, and disrupt expression of gene products (such as M-CSF). When expressed or measured by percentage of base pairs formed, the percentage complementarity that fulfills this goal can range from as little as about 50% complementarity to full (100%) complementary. In general, sufficient complementarity is at least about 50%. In one embodiment, sufficient complementarity is at least about 75% complementarity. In another embodiment, sufficient complementarity is at least about 90% or about 95% complementarity. In yet another embodiment, sufficient complementarity is at least about 98% or 100% complementarity.

A thorough treatment of the qualitative and quantitative considerations involved in establishing binding conditions that allow one skilled in the art to design appropriate oligonucleotides for use under the desired conditions is provided by Beltz et al. Methods Enzymol 100:266-285, 1983, and by Sambrook et al. (ed.), Molecular Cloning: A Laboratory Manual, 2nd ed., vol. 1-3, Cold spring Harbor Laboratory Press, Cold Spring Harbor, N.Y., 1989.

Interefering with or inhibiting (expression of a target gene): This phrase refers to the ability of a siRNA or other molecule to measurably reduce the expression of a target gene. It contemplates reduction of the end-product of the gene, e.g., the expression or function of the encoded protein, and thus includes reduction in the amount or longevity of the mRNA transcript. It is understood that the phrase is relative, and does not require absolute suppression of the gene. Thus, in certain embodiments, interfering with or inhibiting gene expression of a target gene requires that, following application of the dsRNA, the gene is expressed at least 5% less than prior to application of dsDNA, such as at least 10% less, at least 15% less, at least 20% less, at least 25% less, or even more reduced. Thus, in some particular embodiments, application of a dsRNA reduces expression of the target gene by about 30%, about 40%, about 50%, about 60%, or more. In specific examples, where the dsRNA is particularly effective, expression is reduced by 70%, 80%, 85%, 90%, 95%, or even more.

In vitro amplification: Techniques that increase the number of copies of a nucleic acid molecule in a sample or specimen. An example of amplification is the polymerase chain reaction, in which a biological sample collected from a subject is contacted with a pair of oligonucleotide primers, under conditions that allow for the hybridization of the primers to nucleic acid template in the sample. The primers are extended under suitable conditions, dissociated from the template, and then re-annealed, extended, and dissociated to amplify the number of copies of the nucleic acid. The product of in vitro amplification may be characterized by electrophoresis, restriction endonuclease cleavage patterns, oligonucleotide hybridization or ligation, and/or nucleic acid sequencing, using standard techniques. Other examples of in vitro amplification techniques include strand displacement amplification (see U.S. Pat. No. 5,744,311); transcription-free isothermal amplification (see U.S. Pat. No. 6,033,881); repair chain reaction amplification (see WO 90/01069); ligase chain reaction amplification (see EP-A-320 308); gap filling ligase chain reaction amplification (see U.S. Pat. No.5,427,930); coupled ligase detection and PCR (see U.S. Pat. No. 6,027,889); and NASBA™ RNA transcription-free amplification (see U.S. Pat. No.6,025,134).

Isolated: An “isolated” biological component (such as a nucleic acid, peptide or protein) has been substantially separated, produced apart from, or purified away from other biological components in the cell of the organism in which the component naturally occurs, i.e., other chromosomal and extrachromosomal DNA and RNA, and proteins. Nucleic acids, peptides and proteins which have been “isolated” thus include nucleic acids and proteins purified by standard purification methods. The term also embraces nucleic acids, peptides and proteins prepared by recombinant expression in a host cell as well as chemically synthesized nucleic acids.

Nucleostemin: A polypeptide having an amino acid sequence at least 80% identity to SEQ ID NO: 6 which affects the differentiation and/or proliferation of a cell. In one embodiment, a nucleostemin has an amino acid sequence at least 85% identical to SEQ ID NO: 2. In another embodiment, a nucleostemin has an amino acid sequence at least 85% identical to SEQ ID NO: 4. SEQ ID NO: 2 is an exemplary sequence of a mouse nucleostemin, SEQ ID NO: 4 is an exemplary sequence of a rat nucleostemin, and SEQ ID NO: 6 is an exemplary sequence of a human nucleostemin.

Nucleotide: Includes, but is not limited to, a monomer that includes a base linked to a sugar, such as a pyrimidine, purine or synthetic analogs thereof, or a base lined to an amino acid, as in a peptide nucleic acid (PNA). A nucleotide is one monomer in a polynucleotide. A nucleotide sequence refers to the sequence of bases in a polynucleotide.

Operably linked: A first nucleic acid sequence is operably lined with a second nucleic acid sequence when the first nucleic acid sequence is placed in a functional relationship with the second nucleic acid sequence. For instance, a promoter is operably linked to a coding sequence if the promoter affects the transcription or expression of the coding sequence. Generally, operably linked DNA sequences are contiguous and, where necessary to join two protein coding regions, in the same reading frame.

p53: A tumor suppressor gene (for review see Montenarh, Oncogene 7(9):1673-1680, 1992). The p53 gene encodes a 393-amino-acid phosphoprotein that can form complexes with viral proteins such as simian virus 40 (SV40) large-T antigen and adenovirus E1B. Wild-type p53 appears to be important in regulating cell growth and division. Overexpression of wild-type p53 has been shown in some cases to be anti-proliferative in human tumor cell lines. Thus, p53 can act as a negative regulator of cell growth (Hall and Lane, Curr. Biol., 7:R144-R147, 1997) and may directly suppress uncontrolled cell growth or activate genes that suppress this growth. Thus, absence or inactivation of wild-type p53 may contribute to transformation. The protein is found in normal tissues and tumor cells, but at different concentrations. Expression of p53 may be required for normal differentiation in some cell types (Sah et al., Nature Genetics 10:175-180, 1995; Hall and Lane, Curr. Biol., 7:R144-R147, 1997).

High levels of mutant p53 have been found in many cells transformed by chemical carcinogenesis, ultraviolet radiation, and several viruses, including SV40. The p53 gene is a frequent target of mutational inactivation in a wide variety of human tumors and is documented to be frequently-mutated in common human cancers (e.g. see Mercer, Proc. Natl. Acad. Sci. U.S.A. 87:6166-6170, 1990).

Polypeptide: A polymer in which the monomers are amino acid residues which are joined together through amide bonds. When the amino acids are alpha-amino acids, either the L-optical isomer or the D-optical isomer can be used, the L-isomers being preferred. The terms “polypeptide” or “protein” as used herein are intended to encompass any amino acid sequence and include modified sequences such as glycoproteins. The term “polypeptide” is specifically intended to cover naturally occurring proteins, as well as those which are recombinantly or synthetically produced.

The term “polypeptide fragment” refers to a portion of a polypeptide which exhibits at least one useful epitope. The term “functional fragments of a polypeptide” refers to all fragments of a polypeptide that retain an activity of the polypeptide, such as a nucleostemin. Biologically functional fragments, for example, can vary in size from a polypeptide fragment as small as an epitope capable of binding an antibody molecule to a large polypeptide capable of participating in the characteristic induction or programming of phenotypic changes within a cell, including affecting cell proliferation or differentiation. An “epitope” is a region of a polypeptide capable of binding an immunoglobulin generated in response to contact with an antigen. Thus, smaller peptides containing the biological activity of insulin, or conservative variants of the insulin, are thus included as being of use.

The term “soluble” refers to a form of a polypeptide that is not inserted into a cell membrane.

The term “substantially purified polypeptide” as used herein refers to a polypeptide which is substantially free of other proteins, lipids, carbohydrates or other materials with which it is naturally associated. In one embodiment, the polypeptide is at least 50%, for example at least 80% free of other proteins, lipids, carbohydrates or other materials with which it is naturally associated. In another embodiment, the polypeptide is at least 90% free of other proteins, lipids, carbohydrates or other materials with which it is naturally associated. In yet another embodiment, the polypeptide is at least 95% free of other proteins, lipids, carbohydrates or other materials with which it is naturally associated.

Conservative substitutions replace one amino acid with another amino acid that is similar in size, hydrophobicity, etc. Examples of conservative substitutions are shown below:

| Original Residue | Conservative Substitutions |
|---|---|
| Ala | Ser |
| Arg | Lys |
| Asn | Gln, His |
| Asp | Glu |
| Cys | Ser |
| Gln | Asn |
| Glu | Asp |
| His | Asn; Gln |
| Ile | Leu, Val |
| Leu | Ile; Val |
| Lys | Arg; Gln; Glu |
| Met | Leu; Ile |
| Phe | Met; Leu; Tyr |
| Ser | Thr |
| Thr | Ser |
| Trp | Tyr |
| Tyr | Trp; Phe |
| Val | Ile; Leu |

Variations in the cDNA sequence that result in amino acid changes, whether conservative or not, should be minimized in order to preserve the functional and immunologic identity of the encoded protein. Thus, in several non-limiting examples, a nucleostemin polypeptide includes at most two, at most five, at most ten, at most twenty, or at most fifty conservative substitutions. The immunologic identity of the protein may be assessed by determining whether it is recognized by an antibody; a variant that is recognized by such an antibody is immunologically conserved. Any cDNA sequence variant will preferably introduce no more than twenty, and preferably fewer than ten amino acid substitutions into the encoded polypeptide. Variant amino acid sequences may be, for example, at least 80%, 90% or even 95% or 98% identical to the native amino acid sequence.

Pharmaceutically acceptable carriers: The pharmaceutically acceptable carriers of use are conventional. Remington's Pharmaceutical Sciences, by E. W.

Martin, Mack Publishing Co., Easton, Pa., 15th Edition (1975), describes compositions and formulations suitable for pharmaceutical delivery of the fusion proteins herein disclosed.

In general, the nature of the carrier will depend on the particular mode of administration being employed. For instance, parenteral formulations usually comprise injectable fluids that include pharmaceutically and physiologically acceptable fluids such as water, physiological saline, balanced salt solutions, aqueous dextrose, glycerol or the like as a vehicle. For solid compositions (e.g., powder, pill, tablet, or capsule forms), conventional non-toxic solid carriers can include, for example, pharmaceutical grades of mannitol, lactose, starch, or magnesium stearate. In addition to biologically-neutral carriers, pharmaceutical compositions to be administered can contain minor amounts of non-toxic auxiliary substances, such as wetting or emulsifying agents, preservatives, and pH buffering agents and the like, for example, sodium acetate or sorbitan monolaurate.

Pharmaceutical agent: A chemical compound, small molecule, or other composition capable of inducing a desired therapeutic or prophylactic effect when properly administered to a subject or a cell. “Incubating” includes a sufficient amount of time for a drug to interact with a cell. “Contacting” includes incubating a drug in solid or in liquid form with a cell.

Polynucleotide: A nucleic acid sequence (such as a linear sequence) of any length. Therefore, a polynucleotide includes oligonucleotides, and also gene sequences found in chromosomes. An “oligonucleotide” is a plurality of joined nucleotides joined by native phosphodiester bonds. An oligonucleotide is a polynucleotide of between 6 and 300 nucleotides in length. An oligonucleotide analog refers to moieties that function similarly to oligonucleotides but have non-naturally occurring portions. For example, oligonucleotide analogs can contain non-naturally occurring portions, such as altered sugar moieties or inter-sugar linkages, such as a phosphorothioate oligodeoxynucleotide. Functional analogs of naturally occurring polynucleotides can bind to RNA or DNA, and include peptide nucleic acid (PNA) molecules.

Primers: Short nucleic acids, for example DNA oligonucleotides ten nucleotides or more in length, which are annealed to a complementary target DNA strand by nucleic acid hybridization to form a hybrid between the primer and the target DNA strand, then extended along the target DNA strand by a DNA polymerase enzyme. Primer pairs can be used for amplification of a nucleic acid sequence, e.g., by the polymerase chain reaction (PCR) or other nucleic-acid amplification methods known in the art.

Probes and primers as used herein may, for example, include at least 10 nucleotides of the nucleic acid sequences that are shown to encode specific proteins. In order to enhance specificity, longer probes and primers may also be employed, such as probes and primers that comprise 15, 20, 30, 40, 50, 60, 70, 80, 90 or 100 consecutive nucleotides of the disclosed nucleic acid sequences. Methods for preparing and using probes and primers are described in the references, for example Sambrook et al. (1989) Molecular Cloning: A Laboratory Manual, Cold Spring Harbor, N.Y.; Ausubel et al. (1987) Current Protocols in Molecular Biology, Greene Publ. Assoc. & Wiley-Intersciences; Innis et al. (1990) PCR Protocols, A Guide to Methods and Applications, innis et al. (Eds.), Academic Press, San Diego, Calif. PCR primer pairs can be derived from a known sequence, for example, by using computer programs intended for that purpose such as Primer (Version 0.5, 1991, Whitehead Institute for Biomedical Research, Cambridge, Mass.).

When referring to a probe or primer, the term specific for (a target sequence) indicates that the probe or primer hybridizes under stringent conditions substantially only to the target sequence in a given sample comprising the target sequence.

Promoter: A promoter is an array of nucleic acid control sequences which direct transcription of a nucleic acid. A promoter includes necessary nucleic acid sequences near the start site of transcription, such as, in the case of a polymerase II type promoter, a TATA element. A promoter also optionally includes distal enhancer or repressor elements which can be located as much as several thousand base pairs from the start site of transcription.

Recombinant: A recombinant nucleic acid is one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques. Similarly, a recombinant protein is one encoded by a recombinant nucleic acid molecule.

Senescence: The inability of a cell to divide further. A senescent cell is still viable, but does not divide.

Sequence identity: The similarity between amino acid sequences or between nucleic acid sequences can be expressed in terms of the percentage of conservation between the sequences, otherwise referred to as sequence similarity. Sequence identity is frequently measured in terms of percentage identity (or similarity or homology); the higher the percentage, the more similar the two sequences are. Homologues or variants of a nucleostemin (NS) sequence will possess a relatively high degree of sequence identity when aligned using standard methods.

Methods of alignment of sequences for comparison are well known in the art. Various programs and alignment algorithms are described in: Smith and Waterman, Adv. Appl. Math. 2:482, 1981; Needleman and Wunsch, J. Mol. Biol. 48:443, 1970; Pearson and Lipman, Proc. Natl. Acad. Sci. U.S.A. 85:2444, 1988; Higgins and Sharp, Gene 73:237, 1988; Higgins and Sharp, CABIOS 5:151, 1989; Corpet et al., Nucleic Acids Research 16:10881, 1988; and Pearson and Lipman, Proc. Natl. Acad. Sci. U.S.A. 85:2444, 1988. Altschul et al., Nature Genet., 6:119, 1994, presents a detailed consideration of sequence alignment methods and homology calculations.

The NCBI Basic Local Alignment Search Tool (BLAST) (Altschul et al., J. Mol. Biol. 215:403, 1990) is available from several sources, including the National Center for Biotechnology Information (NCBL Bethesda, Md.) and on the Internet, for use in connection with the sequence analysis programs blastp, blastn, blastx, tblastn and tblastx. A description of how to determine sequence identity using this program is available on the NCBI website on the Internet. Other specific, non-limiting examples of sequence alignment programs specifically designed to identify conserved regions of genomic DNA of greater than or equal to 100 nucleotides are PIPMaker (Schwartz et al., Genome Research 10:577-586, 2000) and DOTTER (Erik et al., Gene 167:GC1-10, 1995).

Homologues and variants of a NS sequence are typically characterized by possession of at least 75%, for example at least 80%, 90%, 95%, 98%, or 99%, sequence identity counted over the full length alignment with the originating NS sequence using the NCBI Blast 2.0, set to default parameters. Methods for determining sequence identity over such short windows are available at the NCBI website on the Internet. One of skill in the art will appreciate that these sequence identity ranges are provided for guidance only; it is entirely possible that strongly significant homologues could be obtained that fall outside of the ranges provided.

Small inhibitory RNA (siRNA): Double stranded RNAs (dsRNAs) that can induce gene-specific inhibition of expression in invertebrate and vertebrate species. These RNAs are suitable for interference or inhibition of expression of a target gene and comprise double stranded RNAs of about 15 to about 40 nucleotides containing a 3′ and/or 5′ overhang on each strand having a length of 0- to about 5-nucleotides, wherein the sequence of the double stranded RNAs is substantially identical to a portion of an mRNA or transcript of the target gene for which interference or inhibition of expression is desired. The double stranded RNAs can be formed from complementary ssRNAs or from a single stranded RNA that forms a hairpin or from expression from a DNA vector.

In addition to native RNA molecules, RNA suitable for inhibiting or interfering with the expression of a target nucleostemin sequence include RNA derivatives and analogs. For example, a non-natural linkage between nucleotide residues can be used, such as a phosphorothioate linkage. The RNA strand can be derivatized with a reactive functional group or a reporter group, such as a fluorophore. Particularly useful derivatives are modified at a terminus or termini of an RNA strand, typically the 3′ terminus of the sense strand. For example, the 2′-hydroxyl at the 3′ terminus can be readily and selectively derivatized with a variety of groups.

Other useful RNA derivatives incorporate nucleotides having modified carbohydrate moieties, such as 2′-O-alkylated residues or 2′-deoxy-2′-halogenated derivatives. Particular examples of such carbohydrate moieties include 2′-O-methyl ribosyl derivatives and 2′-O-fluoro ribosyl derivatives.

The RNA bases may also be modified. Any modified base useful for inhibiting or interfering with the expression of a target sequence can be used. For example, halogenated bases, such as 5-bromouracil and 5-iodouracil can be incorporated. The bases can also be alkylated, for example, 7-methylguanosine can be incorporated in place of a guanosine residue. Non-natural bases that yield successful inhibition can also be incorporated.

Stem cell: A cell that can generate a fully differentiated functional cell of more than one given cell type. The role of stem cells in vivo is to replace cells that are destroyed during the normal life of an animal. Generally, stem cells can divide without limit. After division, the stem cell may remain as a stem cell, become a precursor cell, or proceed to terminal differentiation. Although appearing morphologically unspecialized, the stem cell may be considered differentiated where the possibilities for further differentiation are limited. A precursor cell is a cell that can generate a fully differentiated functional cell of at least one given cell type. Generally, precursor cells can divide. After division, a precursor cell can remain a precursor cell, or may proceed to terminal differentiation. In one specific, non-limiting example, a “pancreatic stem cell” is a stem cell of the pancreas. In one embodiment, a pancreatic stem cell gives rise to all of the pancreatic endocrine cells, e.g., the a cells, β cells, δ cells, and PP cells, but does not give rise to other cells such as the pancreatic exocrine cells. A “pancreatic precursor cell” is a precursor cell of the pancreas. In one embodiment, a pancreatic precursor cell gives rise to more than one type of pancreatic endocrine cell. One specific, non-limiting example of a pancreatic precursor cell is a cell that give rise to α and β cells.

Subject: Any mammal, such as humans, non-human primates, pigs, sheep, cows, rodents and the like, which is to be the recipient of the particular treatment. In one embodiment, a subject is a human subject or a murine subject.

Therapeutic agent: Used in a generic sense, it includes treating agents, prophylactic agents, and replacement agents.

Transduced and Transformed: A virus or vector “transduces” a cell when it transfers nucleic acid into the cell. A cell is “transformed” or “transfected” by a nucleic acid transduced into the cell when the DNA becomes stably replicated by the cell, either by incorporation of the nucleic acid into the cellular genome, or by episomal replication.

Numerous methods of transfection are known to those skilled in the art, such as: chemical methods (e.g., calcium-phosphate transfection), physical methods (e.g., electroporation, microinjection, particle bombardment), fusion (e.g., liposomes), receptor-mediated endocytosis (e.g., DNA-protein complexes, viral envelope/capsid-DNA complexes) and by biological infection by viruses such as recombinant viruses (Wolff, J. A., ed, Gene Therapeutics, Birkhauser, Boston, Mass., USA, 1994). In the case of infection by retroviruses, the infecting retrovirus particles are absorbed by the target cells, resulting in reverse transcription of the retroviral RNA genome and integration of the resulting provirus into the cellular DNA. Methods for the introduction of genes into the pancreatic endocrine cells are known (e.g. see U.S. Pat. No. 6,110,743, herein incorporated by reference). These methods can be used to transduce a pancreatic endocrine cell produced by the methods described herein, or an artificial islet produced by the methods described herein.

Genetic modification of the target cell is one indicia of successful transfection. “Genetically modified cells” refers to cells whose genotypes have been altered as a result of cellular uptakes of exogenous nucleotide sequence by transfection. A reference to a transfected cell or a genetically modified cell includes both the particular cell into which a vector or polynucleotide is introduced and progeny of that cell.

Transgene: An exogenous gene supplied by a vector.

Vector: A nucleic acid molecule as introduced into a host cell, thereby producing a transformed host cell. A vector may include nucleic acid sequences that permit it to replicate in the host cell, such as an origin of replication. A vector may also include one or more therapeutic genes and/or selectable marker genes and other genetic elements known in the art. A vector can transduce, transform or infect a cell, thereby causing the cell to express nucleic acids and/or proteins other than those native to the cell. A vector optionally includes materials to aid in achieving entry of the nucleic acid into the cell, such as a viral particle, liposome, protein coating or the like.

Unless otherwise explained, all technical and scientific terms used herein have the same meaning as commonly understood by one of ordinary skill in the art to which this disclosure belongs. The singular terms “a,” “an,” and “the” include plural referents unless context clearly indicates otherwise. Similarly, the word “or” is intended to include “and” unless the context clearly indicates otherwise. It is further to be understood that all base sizes or amino acid sizes, and all molecular weight or molecular mass values, given for nucleic acids or polypeptides are approximate, and are provided for description. Although methods and materials similar or equivalent to those described herein can be used in the practice or testing of this disclosure, suitable methods and materials are described below. The term “comprises” means “includes.”

### Nucleostemin Polypeptides and Polynucleotides

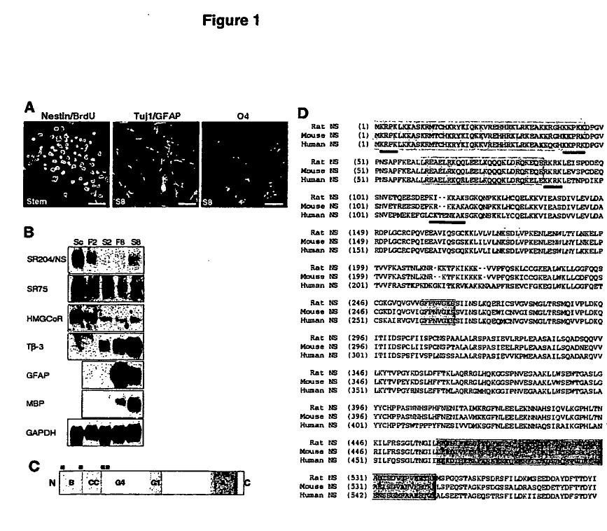

Substantially purified nucleostemin polypeptides are disclosed herein. In one embodiment, a nucleostemin polypeptide has a sequence at least 85% homologous to the amino acid sequence set forth in SEQ ID NO: 2, such as, but not limited to, at least 90%, 95%, or 99% homologous to the amino acid sequence set forth in SEQ ID NO: 2 (see FIG. 1). Thus, in one specific example, a nucleostemin polypeptide has a sequence set forth as SEQ ID NO: 2:

MKRPKLKKASKRMTCHKRYKIQKKVREHHRKLRKEAKKRGHKKPKKDPGV
PNSAPFKEALLREAELRKQQLEELKQQQKLDRQKEQERKRKLEISPDDEQ
SNVETQEESDEPKIKKAKSGKQNPKKLHCQELKKVIEASDIVLEVLDARD
PLGCRCPQVEEAVIQSGCKKLVLVLNKSDLVPKENLENWLTYLNKELPTV
VFKASTNLKNRKKTFKIKKKVVPFQSKLCCGKEALWKLLGGFQQSCGKGV
QVGVVGFPNVGKSSIINSLKQERICSVGVSMGLTRSMQIVPLDKQITIID
SPCFIISPCNSPAALALRSPASIEVLRPLEAASAILSQADSQQVVLKYTV
PGYKDSLDFFTKLAQRRGLHQKGGSPNVESAAKLLWSEWTGASLGYYCHP
PASWNHSPHFNENITAIMKRGFNLEELEKNNAHSIQVLKGPHLTNKILFR
SSGLTNGILEEKDIPEESPKQTEDQQDGDDQEHVTGEKBAEISDVTPVEE
TREMSPGQSTASKPSDRSFILDKMSEEDDAYDFTTDYI

In another embodiment, a nucleostemin polypeptide has a sequence as set forth as SEQ ID NO: 2 or is a conservative variant of SEQ ID NO: 2, such that it includes no more than fifty conservative substitutions of SEQ ID NO: 2, such as no more than two, no more than five, no more than ten, or no more than twenty conservative amino acid substitutions in SEQ ID NO: 2. In another embodiment, a nucleostemin polypeptide has an amino acid sequence as set forth as SEQ ID NO: 2.

In one embodiment, an nucleostemin polypeptide has a sequence at least 85% homologous to the amino acid sequence set forth in SEQ ID NO: 4, such as, but not limited to, at least 90%, 95%, or 99% homologous to the amino acid sequence set forth in SEQ ID NO: 4 (see FIG. 1). In one specific example, a nucleostemin has a sequence set forth as SEQ ID NO: 4:

MKRPKLKKASKRMTCHKRYKIQKKVREHHRKLRKEAKKRGHKKPRKDPGV
PNSAPFKEALLREAELRKQQLEELKQQQKLDRQKEQERKRKLEVSPGDEQ
SNVETREESDEPKRKKAKAGKQNPKKLHCQELKKVIEASDIVLEVLDARD
PLGCRCPQIEEAVIQSGSKKLILVLNKSDLVPKENLENWLNYLNKELPTV
VFKASTNLKNRKTFKIKKKKVVPFQSKICCGKEALWKLLGDFQQSCGKDI
QVGVIGFPNVGKSSVINSLKQEWICNVGISMGLTRSMQIVPLDKQITIID
SPCLIISPCNSPTALALRSPASIEELRPLEAASAILSQADNEQVVLKYTV
PEYKDSLHFFTKLAQRRGLHQKGGSPNVESAAKLVWSEWTGASLGYYCHP
PASWNHSLHFNENIAAVMKKGFNLEELEKNNAHSIQVLKGPHLTNRILFR
SSGLTNGILDEKDIVEESPSQTEDQQDADDQENGSGERNAEISDVAPVEE
TRELSPEQSTAGKPSDGSSALDRASQEDETYDFTTDYI

In a further embodiment, a nucleostemin polypeptide is a conservative variant of SEQ ID NO: 4, such that it includes no more than fifty conservative amino acid substitutions, such as no more than two, no more than five, no more than ten, no more than twenty, or no more than fifty conservative amino acid substitutions in SEQ ID NO: 4. In another embodiment, a nucleostemin polypeptide has an amino acid sequence as set forth as SEQ ID NO: 4.

Specific, non-limiting examples of a nucleostemin polypeptide are conservative variants of SEQ ID NO: 2. A table of conservative substitutions is provided herein. Substitutions of the amino acid sequence shown in SEQ ID NO: 2 can be made based on this table. Thus, one non-limiting example of a conservative variant is substitution of amino acid one (Met) of SEQ ID NO: 2 with an arginine residue. Using the sequence provided as SEQ ID NO: 2, and the description of conservative amino acid substitutions provided, one of skill in the art can readily ascertain sequences of conservative variants. In several embodiments, a conservative variant includes at most one, at most two, at most five, at most ten, or at most fifteen conservative substitutions of the sequence shown in SEQ ID NO: 2. Generally, a conservative variant will bind to antibodies that immunoreact with a polypeptide including a sequence set forth as SEQ ID NO: 2, and/or will immunoreact with a polypeptide including a sequence set forth as SEQ ID NO: 4.

Fragments and variants of a nucleostemin polypeptide can readily be prepared by one of skill in the art using molecular techniques. In one embodiment, a fragment of a nucleostemin polypeptide includes at least 8, 10, 15, or 20 consecutive amino acids of the nucleostemin polypeptide. In another embodiment, a fragment of a nucleostemin polypeptide includes a specific antigenic epitope found on a full-length nucleostemin. In a further embodiment, a fragment of nucleostemin is a fragment that confers a function of nucleostemin when transferred into a cell of interest, such as, but not limited to, inducing differentiation or decreasing proliferation of the cell.

One skilled in the art, given the disclosure herein, can purify a nucleostemin polypeptide using standard techniques for protein purification. The substantially pure polypeptide will yield a single major band on a non-reducing polyacrylamide gel. The purity of the nucleostemin polypeptide can also be determined by amino-terminal amino acid sequence analysis.

Minor modifications of the nucleostemin polypeptide primary amino acid sequences may result in peptides which have substantially equivalent activity as compared to the unmodified counterpart polypeptide described herein. Such modifications may be deliberate, as by site-directed mutagenesis, or may be spontaneous. All of the polypeptides produced by these modifications are included herein.

One of skill in the art can readily produce fusion proteins including a nucleostemin polypeptide and a second polypeptide of interest. Optionally, a linker can be included between the nucleostemin polypeptide and the second polypeptide of interest. Fusion proteins include, but are not limited to, a polypeptide including a nucleostemin polypeptide and a marker protein. In one embodiment, the marker protein can be used to identify or purify a nucleosterin polypeptide. Exemplary fusion proteins include, but are not limited to, green fluorescent protein, six histidine residues, or myc and a nucleostemin polypeptide.

As disclosed herein, an increase or decrease in the concentration of nucleostemin polypeptides induces differentiation of cells, such as, but not limited to, stem cells. An increase or decrease in the concentration of nucleostemin polypeptides inhibits proliferation of cells, such as, but not limited to, stem cells. In one specific non-limiting example, functional nucleostemin polypeptides include a CC domain such as an amino acid sequence 100% identical to one of (a) amino acids 61-87 of SEQ ID NO: 2, (b) amino acids 61-87 of SEQ ID NO: 4, or (c) amino acids 61-87 of SEQ ID NO: 6. In another specific, non-limiting example, a functional nucleostemin polypeptide includes a G4 motif, such as an amino acid sequence 100% identical to one of (a) amino acids 177-180 of SEQ ID NO: 2, (b) amino acids 177-180 of SEQ ID NO: 4, or (c) amino acids 177-180 of SEQ ID NO: 6. In a further specific, non-limiting example, a functional nucleostemin polypeptide includes a G1 motif such as an amino acid sequence 100% identical to one of (a) amino acids 256-263 of SEQ ID NO: 2, (b) amino acids 256-263 of SEQ ID NO: 4, or (c) amino acids 256-263 of SEQ ID NO: 6. Functional nucleostemin polypeptides also include at least two of a CC domain, a G1 domain, or a G4 domain, or include all three of a CC domain, a G1 domain, and a G4 domain.

Polynucleotides encoding a nucleostemin polypeptide are also provided, and are termed nucleostemin polynucleotides. These polynucleotides include DNA, cDNA and RNA sequences which encode a nucleostemin. It is understood that all polynucleotides encoding a nucleostemin polypeptide are also included herein, as long as they encode a polypeptide with the recognized activity, such as the binding to an antibody that recognizes a nucleostemin polypeptide, or modulating cellular differentiation or proliferation. The polynucleotides include sequences that are degenerate as a result of the genetic code. There are 20 natural amino acids, most of which are specified by more than one codon. Therefore, all degenerate nucleotide sequences are included as long as the amino acid sequence of the nucleostemin polypeptide encoded by the nucleotide sequence is functionally unchanged. One specific, non-limiting example of a polynucleotide encoding a nucleostemin is SEQ ID NO: 1, as set forth below:

GCGGCCGAGGTACCTGAGACCTCTTCTGCTCCAGACGCGTCCGCGGCCAG
GATGAAGAGGCCGAAGTTAAAGAAAGCAAGTAAACGTATGACCTGTCATA
AGCGGTATAAAATCCAGAAAAAGGTTCGAGAACATCATCGAAAATTAAGG
AAGGAAGCTAAAAAGCGGGGTCACAAGAAGCCTAAGAAGGACCCAGGAGT
TCCAAATAGTGCTCCCTTTAAAGAGGCTCTTCTTCGTGAAGCTGAGCTAA
GGAAACAGCAGCTTGAAGAACTAAAACAGCAGCAGAAACTTGACAGGCAA
AAAGAACAAGAACGAAAAAGAAAACTTGAAATTAGCCCTGATGATGAGCA
ATCTAATGTGGAAACTCAGGAGGAATCTGATGAGCCCAAAATAAAGAAAG
CTAAATCAGGCAAACAGAATCCAAAGAAGTTACATTGTCAGGAACTTAAA
AAGGTGATTGAAGCCTCAGACATTGTGTTAGAAGTTTTGGATGCCAGAGA
TCCTCTTGGTTGCAGGTGTCCTCAAGTAGAAGAAGCTGTTATCCAAAGTG
GATGTAAAAAACTAGTACTTGTATTAAATAAGTCAGATCTAGTACCAAAA
GAGAATCTAGAGAACTGGCTAACTTACTTGAATAAGGAATTGCCAACAGT
GGTGTTCAAAGCCTCAACAAACTTAAAGAACAGAAAGAAGACATTCAAGA
TAAAGAAGAAAGTTGTTCCATTCCAAAGTAAACTCTGCTGTGGCAAGGAA
GCACTGTGGAAGCTCCTTGGAGGTTTTCAGCAGTCCTGTGGAAAAGGAGT
TCAGGTTGGAGTGGTTGGTTTCCCAAATGTGGGAAAAAGCAGCATCATTA
ATAGTTTAAAACAAGAAAGGATTTGCAGTGTTGGAGTTTCCATGGGACTT
ACAAGGAGTATGCAGATTGTCCCTTTAGACAAACAGATCACAATCATAGA
TAGTCCGTGCTTCATTATCTCACCTTGTAACTCCCCTGCTGCACTTGCCC
TCCGAAGTCCAGCAAGTATTGAAGTTCTAAGACCATTGGAGGCTGCCAGT
GCCATCCTGTCTCAGGCTGATAGTCAACAGGTGGTGTTAAAATATACTGT
CCCGGGGTATAAGGATTCTCTGGATTTTTTTACTAAACTTGCTCAGAGAA
GAGGTCTGCACCAAAAAGGTGGAAGCCCAAATGTCGAAAGTGCTGCTAAG
CTGCTATGGTCTGAGTGGACAGGTGCCTCATTAGGTTACTACTGCCATCC
CCCTGCATCCTGGAATCATTCTCCTCATTTTAATGAGAATATTACAGCAA
TCATGAAGAGGGGCTTTAATCTAGAAGAACTAGAAAAGAATAATGCACAC
AGCATACAAGTCCTCAAGGGCCCTCATTTAACTAATAAAATCCTTTTCCG
GTCTTCGGGCCTGACAAATGGAATACTAGAAGAAAAGGACATCCCCGAAG
AGTCACCAAAACAGACAGAAGACCAACAGGATGGTGATGATCAAGAACAT
GTTACTGGTGAAAAAAATGCAGAGATCTCAGATGTGACTCCTGTAGAAGA
GACCAGGGAGATGTCACCTGGGCAATCAACAGCAAGTAAACCATCTGACA
GATCCTTTATCTTGGATAAAATGAGTGAAGAAGACGATGCCTATGACTTT
ACCACAGATTATATATAGCCTTCTAAATGTTCAAGTGTGCTCTGTACAGT
GTTTNTAGATTGCTTTGGTATGATATAAAGTGTAAATCTTGTGAATATGT
ATCATGTTTTAAATTAAAAACAAAATAAAAAGTGTTTGTATAAAAAAAAA
AAAAAAAAAA

Another specific non-limiting example of a polynucleotide encoding a nucleostemin is a polynucleotide having at least 85% homology to SEQ ID NO: 1, such as a polynucleotide at least 90%, 95%, or 99% homologous to SEQ ID NO: 1 that encodes a polypeptide having an antigenic epitope or function of a nucleostemin. Yet another specific non-limiting example of a polynucleotide encoding a nucleostemin is a polynucleotide that encodes a polypeptide that is specifically bound by an antibody that specifically binds SEQ ID NO: 2.

Other specific, non-limiting example of a polynucleotide encoding a nucleostemin is SEQ ID NO: 3, as set forth below:

GAATTCGGCACGAGGGTTGAACCGCAGTTCCAGTTCGCACGTGGCGCCCG
AGAAGTCGTGGTGATCCCGAGACCTCCTCTGCTCCTGAAGCGTCCGCGGC
CAGGATGAAGAGGCCTAAGTTAAAGAAAGCGAGTAAACGTATGACCTGCC
ATAAGCGATATAAAATTCAAAAAAAGGTCCGAGAACATCATCGAAAATTA
AGGAAGGAAGCTAAAAAACGGGGTCACAAGAAGCCTAGGAAGGACCCAGG
TGTTCCAAATAGTGCTCCCTTTAAAGAGGCTCTTCTTCGTGAAGCTGAAC
TAAGGAAACAGCAGCTTGAAGAACTAAAACAGCAGCAGAAACTTGATAGG
CAAAAAGAGCAAGAAAGGAAAAGAAAACTTGAAGTTAGCCCTGGTGATGA
GCAGTCTAATGTGGAAACTAGGGAGGAATCTGACGAGCCCAAAAGAAAGA
AAGCCAAAGCAGGCAAACAGAATCCAAAGAAGTTACATTGCCAGGAACTT
AAAAAGGTGATTGAAGCCTCAGACATTGTGTTAGAAGTTTTGGATGCCAG
AGATCCTCTTGGTTGCAGGTGTCCTCAGATAGAAGAAGCTGTTATCCAGA
GTGGGAGTAAGAAGCTGATACTTGTATTAAATAAGTCTGATCTAGTACCA
AAGGAGAATTTGGAGAACTGGCTAAATTATTTGAATAAAGAATTGCCAAC
CGTGGTGTTCAAAGCCTCAACAAACTTAAAGAACAGAAAGACATTCAAGA
TAAAAAAGAAGAAAGTTGTTCCATTCCAAAGCAAAATCTGCTGTGGCAAG
GAAGCCCTTTGGAAGCTTCTTGGAGATTTCAGCAGTCCTGTGGAAAGGAT
AATTCAAGTTGGAGTGATTGGTTTCCCAAATGTGGGGAAAAGCAGTGTCA
TTAATAGCTTAAAACAAGAATGGATTTGCAATGTTGGGATTTCCATGGGA
CTTACAAGGAGCATGCAGATTGTCCCTTTAGACAAGCAGATCACAATCAT
AGACAGTCCATGCCTAATTATCTCACCTTGTAACTCCCCCACTGCACTTG
CCCTTCGGAGTCCAGCAAGCATTGAGGAACTAAGACCGCTGGAGGCTGCC
AGTGCCATTCTGTCTCAGGCTGATAATGAACAGGTGGTGTTAAAATATAC
TGTCCCTGAGTATAAGGATTCTCTGCATTTTTTTACTAAACTTGCTCAAA
GAAGAGGTCTGCACCAAAAAGGTGGAAGCCCAAATGTGGAAAGTGCTGCT
AAGCTGGTGTGGTCTGAGTGGACAGGTGCCTCAATAGGTTACTATTGCCA
TCCCCCTGCATCCTGGAATCATTCTCTGCATTTTAATGAGAATAATGCAG
CAGTCATGAAGAAGGGCTTTAATCTAGAAGAACTAGAAAAGAATAATGCA
CACAGCATACAAGTCCTCAAGGGCCCTCATTTAACTAATAGAATCCTTTT
TCGGTCTTCGGGCCTGACAAATGGAATACTAGACGAGAAGGACATAGTCG
AAGAGACCAGGGAGCTGTCACCTGAGCAATCAACAGCAGGTAAGCCATCT
GACGGGTCGTCTGCCTTGGATAGAGCGAGTCAAGAGGATGAAACCTATGA
CTTCACCACAGATTATATATAACCGCCACACACTAACGTGCTCTCTGTAC
GCTGTGTAGTTTAGTGTATGATATAAACTGTACATCTTGTAAATATGTAT
CATGTTATAAATTCAAATAAATACAAGTATTTGCTTGCAAAAAAAAAAAA
AAAAAAAACTCGACTCTAGA

Another specific non-limiting example of a polynucleotide encoding a nucleostemin is a polynucleotide having at least 85% homology to SEQ ID NO: 3, such as a polynucleotide at least 90%, 95%, or 99% homologous to SEQ ID NO: 3 that encodes a polypeptide having an antigenic epitope or function of a nucleostemin. Yet another specific non-limiting example of a polynucleotide encoding a nucleostemin is a polynucleotide that encodes a polypeptide that is specifically bound by an antibody that specifically binds SEQ ID NO: 4.

The nucleostemin polynucleotides include a recombinant DNA which is incorporated into a vector; into an autonomously replicating plasmid or virus; or into the genomic DNA of a prokaryote or eukaryote, or which exists as a separate molecule (e.g., a cDNA) independent of other sequences. The nucleotides can be ribonucleotides, deoxyribonucleotides, or modified forms of either nucleotide. The term includes single and double forms of DNA. Also included in this disclosure are fragments of the above-described nucleic acid sequences that are at least 15 bases in length, which is sufficient to permit the fragment to selectively hybridize to DNA that encodes the disclosed nucleostemin polypeptide (e.g., a polynucleotide that encodes SEQ ID NO: 2 or SEQ ID NO: 4) under physiological conditions. The term “selectively hybridize” refers to hybridization under moderately or highly stringent conditions, which excludes non-related nucleotide sequences. The nucleostemin polynucleotide sequences disclosed herein include, but are not limited to, sequences that encode SEQ ID NO: 2, degenerate variants that encode SEQ ID NO: 2, sequences that encode SEQ ID NO: 4, degenerate variants that encode SEQ ID NO: 4, and sequences that encode conservative variations of SEQ ID NO: 2 and/or SEQ ID NO: 4.

### Expression Systems

A polynucleotide encoding nucleosteniin can be included in an expression vector to direct expression of the nucleostemin nucleic acid sequence. Thus, other expression control sequences including appropriate promoters, enhancers, transcription terminators, a start codon (i.e., ATG) in front of a protein-encoding gene, splicing signal for introns, maintenance of the correct reading frame of that gene to permit proper translation of mRNA, and stop codons can be included with a NS sequence in an expression vector. Generally expression control sequences include a promoter, a minimal sequence sufficient to direct transcription.

The expression vector typically contains an origin of replication, a promoter, as well as specific genes which allow phenotypic selection of the transformed cells (e.g. an antibiotic resistance cassette). Vectors suitable for use include, but are not limited, to the pMSXND expression vector for expression in mammalian cells (Lee and Nathans, J. Biol. Chem. 263:3521, 1988). Generally, the expression vector will include a promoter. The promoter can be inducible or constitutive. The promoter can be tissue specific. Suitable promoters include the thymidine kinase promoter (TK), metallothionein I, polyhedron, neuron specific enolase, thyrosine hyroxylase, beta-actin, or other promoters. In one embodiment, the promoter is a heterologous promoter.

In one example, the polynucleotide encoding nucleostemin is located downstream of the desired promoter. Optionally, an enhancer element is also included, and can generally be located anywhere on the vector and still have an enhancing effect. However, the amount of increased activity will generally diminish with distance.

Expression vectors including a polynucleotide encoding nucleostemin can be used to transform host cells. Hosts can include isolated microbial, yeast, insect and mammalian cells, as well as cells located in the organism. Biologically functional viral and plasmid DNA vectors capable of expression and replication in a host are known in the art, and can be used to transfect any cell of interest. Where the cell is a mammalian cell, the genetic change is generally achieved by introduction of the DNA into the genome of the cell (i.e., stable) or as an episome.

A “transfected cell” is a cell into which (or into an ancestor of which) has been introduced, by means of recombinant DNA techniques, a DNA molecule encoding nucleostemin. Transfection of a host cell with recombinant DNA may be carried out by conventional techniques as are well known to those skilled in the art. Where the host is prokaryotic, such as E. coli, competent cells which are capable of DNA uptake can be prepared from cells harvested after exponential growth phase and subsequently treated by the CaCl₂ method using procedures well known in the art. Alternatively, MgCl₂ or RbCl can be used. Transformation can also be performed after forming a protoplast of the host cell if desired, or by electroporation.

When the host is a eukaryote, such methods of transfection of DNA as calcium phosphate co-precipitates, conventional mechanical procedures such as microinjection, electroporation, insertion of a plasmid encased in liposomes, or virus vectors may be used. Eukaryotic cells can also be cotransformed with DNA sequences encoding nucleostemin, and a second foreign DNA molecule encoding a selectable phenotype, such as neomycin resistance. Another method is to use a eukaryotic viral vector, such as simian virus 40 (SV40) or bovine papilloma virus, to transiently infect or transform eukaryotic cells and express the protein (see for example, Eukaryotic Viral Vectors, Cold Spring Harbor Laboratory, Gluzman ed., 1982). Other specific, non-limiting examples of viral vectors include adenoviral vectors, lentiviral vectors, retroviral vectors, and pseudorabies vectors.

The nucleostemin polynucleotide sequences disclosed herein can also be used in the production of transgenic animals such as transgenic mice, as described below.

In one embodiment, a non-human animal is generated that carries a transgene comprising a nucleic acid encoding a nucleostemin operably linked to a promoter. Specific promoters of use include, but are not limited to, a tissue specific promoter such as, but not limited to, an immunoglobulin promoter, a neuronal specific promoter, or the insulin promoter. Specific promoters of use also include a constitutive promoter, such as, but not limited to, the thymdine kinase promoter or the human β-globin minimal, or an actin promoter, amongst others.

This construct can be introduced into a vector to produce a product that is then amplified, for example, by preparation in a bacterial vector, according to conventional methods (see, for example, Sambrook et al, Molecular Cloning: a Laboratory Manual, Cold Spring Harbor Press, 1989). The amplified construct is thereafter excised from the vector and purified for use in producing transgenic animals.

Any transgenic animal can be of use in the methods disclosed herein, provided the transgenic animal is a non-human animal. A “non-human animal” includes, but is not limited to, a non-human primate, a farm animal such as swine, cattle, and poultry, a sport animal or pet such as dogs, cats, horses, hamsters, rodents, or a zoo animal such as lions, tigers or bears. In one specific, non-limiting example, the non-human animal is a transgenic animal, such as, but not limited to, a transgenic mouse, cow, sheep, or goat. In one specific, non-limiting example, the transgenic animal is a mouse. In a particular example, the transgenic animal has altered proliferation and/or differentiation of a cell type as compared to a non-transgenic control (wild-type) animal of the same species.

A transgenic animal contains cells that bear genetic information received, directly or indirectly, by deliberate genetic manipulation at the subcellular level, such as by microinjection or infection with a recombinant virus, such that a recombinant DNA is included in the cells of the animal. This molecule can be integrated within the animal's chromosomes, or can be included as extrachromosomally replicating DNA sequences, such as might be engineered into yeast artificial chromosomes. A transgenic animal can be a “germ cell line” transgenic animal, such that the genetic information has been taken up and incorporated into a germ line cell, therefore conferring the ability to transfer the information to offspring. If such offspring in fact possess some or all of that information, then they, too, are transgenic animals.

Transgenic animals can readily be produced by one of skill in the art. For example, transgenic animals can be produced by introducing into single cell embryos DNA encoding a marker, in a manner such that the polynucleotides are stably integrated into the DNA of germ line cells of the mature animal and inherited in normal Mendelian fashion. Advances in technologies for embryo micromanipulation permit introduction of heterologous DNA into fertilized mammalian ova. For instance, totipotent or pluripotent stem cells can be transformed by microinjection, calcium phosphate mediated precipitation, liposome fusion, retroviral infection or other means. The transformed cells are then introduced into the embryo, and the embryo then develops into a transgenic animal. In one non-limiting method, developing embryos are infected with a retrovirus containing the desired DNA, and a transgenic animal is produced from the infected embryo.

In another specific, non-limiting example, the appropriate DNA(s) are injected into the pronucleus or cytoplasm of embryos, preferably at the single cell stage, and the embryos are allowed to develop into mature transgenic animals. These techniques are well known. For instance, reviews of standard laboratory procedures for microinjection of heterologous DNAs into mammalian (mouse, pig, rabbit, sheep, goat, cow) fertilized ova include: Hogan et al., Manipulating the Mouse Embryo, Cold Spring Harbor Press, 1986; Krimpenfort et al., Bio/Technology 9:86, 1991; Palmiter et al., Cell 41:343, 1985; Kraemer et al., Genetic Manipulation of the Early Mammalian Embryo, Cold Spring Harbor Laboratory Press, 1985; Hammer et al., Nature 315:680, 1985; Purcel et al., Science 244:1281, 1986; U.S. Pat. No. 5,175,385; U.S. Pat. No. 5,175,384.

### Antibodies

A nucleostemin polypeptide or a fragment or conservative variant thereof can be used to produce antibodies which are immunoreactive or bind to an epitope of a nucleostemin. Polyclonal antibodies, antibodies which consist essentially of pooled monoclonal antibodies with different epitopic specificities, as well as distinct monoclonal antibody preparations are included.

The preparation of polyclonal antibodies is well known to those skilled in the art. See, for example, Green et al., “Production of Polyclonal Antisera,” in: Immunochemical Protocols, pages 1-5, Manson, ed., Humana Press, 1992; Coligan et al., “Production of Polyclonal Antisera in Rabbits, Rats, Mice and Hamsters,” in: Current Protocols in Immunology, section 2.4.1, 1992.

The preparation of monoclonal antibodies likewise is conventional. See, for example, Kohler & Milstein, Nature 256:495, 1975; Coligan et al., sections 2.5.1-2.6.7; and Harlow et al. in: Antibodies: a Laboratory Manual, page 726, Cold Spring Harbor Pub., 1988. Briefly, monoclonal antibodies can be obtained by injecting mice with a composition comprising an antigen, verifying the presence of antibody production by removing a serum sample, removing the spleen to obtain B lymphocytes, fusing the B lymphocytes with myeloma cells to produce hybridomas, cloning the hybridomas, selecting positive clones that produce antibodies to the antigen, and isolating the antibodies from the hybridoma cultures. Monoclonal antibodies can be isolated and purified from hybridoma cultures by a variety of well-established techniques. Such isolation techniques include affinity chromatography with Protein-A Sepharose, size-exclusion chromatography, and ion-exchange chromatography. See, e.g., Coligan et al., sections 2.7.1-2.7.12 and sections 2.9.1-2.9.3; Barnes et al., Purification of Immunoglobulin G (IgG), in: Methods in Molecular Biology, Vol. 10, pages 79-104, Humana Press, 1992.

Methods of in vitro and in vivo multiplication of monoclonal antibodies are well known to those skilled in the art. Multiplication in vitro may be carried out in suitable culture media such as Dulbecco's Modified Eagle Medium or RPMI 1640 medium, optionally supplemented by a mammalian serum such as fetal calf serum or trace elements and growth-sustaining supplements such as normal mouse peritoneal exudate cells, spleen cells, thymocytes or bone marrow macrophages. Production in vitro provides relatively pure antibody preparations and allows scale-up to yield large amounts of the desired antibodies. Large-scale hybridoma cultivation can be carried out by homogenous suspension culture in an airlift reactor, in a continuous stirrer reactor, or in immobilized or entrapped cell culture. Multiplication in vivo may be carried out by injecting cell clones into mammals histocompatible with the parent cells, e.g., syngeneic mice, to cause growth of antibody-producing tumors. Optionally, the animals are primed with a hydrocarbon, especially oils such as pristane (tetramethylpentadecane) prior to injection. After one to three weeks, the desired monoclonal antibody is recovered from the body fluid of the animal.

Antibodies can also be derived from a subhuman primate antibody. General techniques for raising therapeutically useful antibodies in baboons can be found, for example, in WO 91/11465, 1991, and Losman et al., Int. J. Cancer 46:310, 1990.

Alternatively, an antibody that specifically binds a nucleostemin polypeptide can be derived from a humanized monoclonal antibody. Humanized monoclonal antibodies are produced by transferring mouse complementarity determining regions from heavy and light variable chains of the mouse immunoglobulin into a human variable domain, and then substituting human residues in the framework regions of the murine counterparts. The use of antibody components derived from humanized monoclonal antibodies obviates potential problems associated with the immunogenicity of murine constant regions. General techniques for cloning murine immunoglobulin variable domains are described, for example, by Orlandi et al., Proc. Natl. Acad Sci. U.S.A. 86:3833, 1989. Techniques for producing humanized monoclonal antibodies are described, for example, by Jones et al., Nature 321:522, 1986; Riechmann et al., Nature 332:323, 1988; Verhoeyen et al., Science 239:1534, 1988; Carter et al., Proc. Natl. Acad. Sci. U.S.A. 89:4285, 1992; Sandhu, Crit. Rev. Biotech. 12:437, 1992; and Singer et al., J. Immunol. 150:2844, 1993.

Antibodies can be derived from human antibody fragments isolated from a combinatorial immunoglobulin library. See, for example, Barbas et al., in: Methods: a Companion to Methods in Enzymology, Vol. 2, page 119, 1991; Winter et al., Ann. Rev. Immunol. 12:433, 1994. Cloning and expression vectors that are useful for producing a human immunoglobulin phage library can be obtained, for example, from STRATAGENE Cloning Systems (La Jolla, Calif.).

In addition, antibodies can be derived from a human monoclonal antibody. Such antibodies are obtained from transgenic mice that have been “engineered” to produce specific human antibodies in response to antigenic challenge. In this technique, elements of the human heavy and light chain loci are introduced into strains of mice derived from embryonic stem cell lines that contain targeted disruptions of the endogenous heavy and light chain loci. The transgenic mice can synthesize human antibodies specific for human antigens, and the mice can be used to produce human antibody-secreting hybridomas. Methods for obtaining human antibodies from transgenic mice are described by Green et al., Nature Genet. 7:13, 1994; Lonberg et al., Nature 368:856, 1994; and Taylor et al., Int. Immunol. 6:579, 1994.

Antibodies include intact molecules as well as fragments thereof, such as Fab, F(ab′)₂, and Fv which are capable of binding the epitopic determinant. These antibody fragments retain some ability to selectively bind with their antigen or receptor and are defined as follows:

(1) Fab, the fragment which contains a monovalent antigen-binding fragment of an antibody molecule, can be produced by digestion of whole antibody with the enzyme papain to yield an intact light chain and a portion of one heavy chain;

(2) Fab′, the fragment of an antibody molecule can be obtained by treating whole antibody with pepsin, followed by reduction, to yield an intact light chain and a portion of the heavy chain; two Fab′ fragments are obtained per antibody molecule;

(3) (Fab′)₂, the fragment of the antibody that can be obtained by treating 25 whole antibody with the enzyme pepsin without subsequent reduction; F(ab′)₂ is a dimer of two Fab′ fragments held together by two disulfide bonds;

(4) Fv, defined as a genetically engineered fragment containing the variable region of the light chain and the variable region of the heavy chain expressed as two chains; and

(5) Single chain antibody (SCA), defined as a genetically engineered molecule containing the variable region of the light chain, the variable region of the heavy chain, linked by a suitable polypeptide linker as a genetically fused single chain molecule.

Methods of making these fragments are known in the art (see for example, Harlow and Lane, Antibodies: A Laboratory Manual, Cold Spring Harbor Laboratory, New York, 1988). An epitope is any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

Antibody fragments can be prepared by proteolytic hydrolysis of the antibody or by expression in E. coli of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)₂. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly (see U.S. Pat. No. 4,036,945 and U.S. Pat. No. 4,331,647, and references contained therein; Nisonhoff et al., Arch. Biochem. Biophys. 89:230, 1960; Porter, Biochem. J. 73:119, 1959; Edelman et al., Methods in Enzymology, Vol. 1, page 422, Academic Press, 1967; and Coligan et al. at sections 2.8.1-2.8.10 and 2.10.1-2.10.4).

Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

For example, Fv fragments comprise an association of V_{H} and V_{L} chains. This association may be noncovalent (Inbar et al., Proc. Natl Acad. Sci. U.S.A. 69:2659, 1972). Alternatively, the variable chains can be linked by an intermolecular disulfide bond or cross-linked by chemicals such as glutaraldehyde. See, e.g., Sandhu, supra. Preferably, the Fv fragments comprise V_{H} and V_{L} chains connected by a peptide linker. These single-chain antigen binding proteins (sFv) are prepared by constructing a structural gene comprising DNA sequences encoding the V_{H} and V_{L} domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are known in the art (see Whitlow et al., Methods: a Companion to Methods in Enzymology, Vol. 2, page 97, 1991; Bird et al., Science 242:423, 1988; U.S. Pat. No. 4,946,778; Pack et al., Bio/Technology 11:1271, 1993; and Sandhu, supra).

Another form of an antibody fragment is a peptide coding for a single complementarity-determining region (CDR). CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells (Larrick et al., Methods: a Companion to Methods in Enzymnology, Vol. 2, page 106, 1991).

Antibodies can be prepared using an intact polypeptide or fragments containing small peptides of interest as the immunizing antigen. The polypeptide or a peptide used to immunize an animal can be derived from substantially purified polypeptide produced in host cells, in vitro translated cDNA, or chemical synthesis which can be conjugated to a carrier protein, if desired. Such commonly used carriers which are chemically coupled to the peptide include keyhole limpet hemocyanin (KLH), thyroglobulin, bovine serum albumin (BSA), and tetanus toxoid. The coupled peptide is then used to immunize the animal (e.g., a mouse, a rat, or a rabbit).

Polyclonal or monoclonal antibodies can be further purified, for example, by binding to and elution from a matrix to which the polypeptide or a peptide to which the antibodies were raised is bound. Those of skill in the art will know of various techniques common in the immunology arts for purification and/or concentration of polyclonal antibodies, as well as monoclonal antibodies (see, for example, Coligan et al., Unit 9, Current Protocols in Immunology, Wiley Interscience, 1991).

It is also possible to use the anti-idiotype technology to produce monoclonal antibodies which mimic an epitope. For example, an anti-idiotypic monoclonal antibody made to a first monoclonal antibody will have a binding domain in the hypervariable region that is the “image” of the epitope bound by the first monoclonal. antibody.

Binding affinity for a target antigen is typically measured or determined by standard antibody-antigen assays, such as competitive assays, saturation assays, or immunoassays such as ELISA or RIA. Such assays can be used to determine the dissociation constant of the antibody. The phrase “dissociation constant” refers to the affinity of an antibody for an antigen. Specificity of binding between an antibody and an antigen exists if the dissociation constant (K_{D}=1/K, where K is the affinity constant) of the antibody is, for example <1 μM, <100 nM, or <0.1 nM. Antibody molecules will typically have a K_{D} in the lower ranges. K_{D}=[Ab-Ag]/[Ab][Ag] where [Ab] is the concentration at equilibrium of the antibody, [Ag] is the concentration at equilibrium of the antigen and [Ab-Ag] is the concentration at equilibrium of the antibody-antigen complex. Typically, the binding interactions between antigen and antibody include reversible noncovalent associations such as electrostatic attraction, Van der Waals forces and hydrogen bonds.

Effector molecules, e.g., therapeutic, diagnostic, or detection moieties can be linked to an antibody that specifically binds nucleostemin, using any number of means known to those of skill in the art. Exemplary effector molecules include, but not limited to, radiolabels, fluorescent markers, or toxins (e.g. Pseudomonas exotoxin (PE), see “Monoclonal Antibody-Toxin Conjugates: Aiming the Magic Bullet,” Thorpe et al., “Monoclonal Antibodies in Clinical Medicine,” Academic Press, pp. 168-190, 1982; Waldmann, Science, 252: 1657, 1991; U.S. Pat. No. 4,545,985 and U.S. Pat. No. 4,894,443, for a discussion of toxins and conjugation). Both covalent and noncovalent attachment means may be used. The procedure for attaching an effector molecule to an antibody varies according to the chemical structure of the effector. Polypeptides typically contain a variety of functional groups; e.g., carboxylic acid (COOH), free amine (—NH₂) or sulfhydryl (—SH) groups, which are available for reaction with a suitable functional group on an antibody to result in the binding of the effector molecule. Alternatively, the antibody is derivatized to expose or attach additional reactive functional groups. The derivatization may involve attachment of any of a number of linker molecules such as those available from Pierce Chemical Company, Rockford, Ill. The linker can be any molecule used to join the antibody to the effector molecule. The linker is capable of forming covalent bonds to both the antibody and to the effector molecule. Suitable linkers are well known to those of skill in the art and include, but are not limited to, straight or branched-chain carbon linkers, heterocyclic carbon linkers, or peptide linkers. Where the antibody and the effector molecule are polypeptides, the linkers may be joined to the constituent amino acids through their side groups (e.g., through a disulfide linkage to cysteine) or to the alpha carbon amino and carboxyl groups of the terminal amino acids.

In some circumstances, it is desirable to free the effector molecule from the antibody when the immunoconjugate has reached its target site. Therefore, in these circumstances, immunoconjugates will comprise linkages that are cleavable in the vicinity of the target site. Cleavage of the linker to release the effector molecule from the antibody may be prompted by enzymatic activity or conditions to which the immunoconjugate is subjected either inside the target cell or in the vicinity of the target site. When the target site is a tumor, a linker which is cleavable under conditions present at the tumor site (e.g., when exposed to tumor-associated enzymes or acidic pH) may be used.

In view of the large number of methods that have been reported for attaching a variety of radiodiagnostic compounds, radiotherapeutic compounds, label (e.g. enzymes or fluorescent molecules) drugs, toxins, and other agents to antibodies, one skilled in the art will be able to determine a suitable method for attaching a given agent to an antibody or other polypeptide.

### Methods of Inducing Differentiation and/or Inhibiting Proliferation

A method for inducing differentiation of a cell is disclosed herein. A method for inhibiting proliferation is also disclosed herein. A method is also provided herein for inducing senescence of a cell. The methods include altering the level of a nucleostemin polypeptide in the cell, thereby inducing differentiation and/or inhibiting proliferation of the cell, and/or inducing senescence of the cell. The cell can be in vivo or in vitro.

Expression of nucleostemin can be either increased or decreased to induce differentiation and/or inhibit proliferation. In one example, expression of nucleostemin is increased as compared to a control. Increased expression includes, but is not limited to, at least a 20% increase in the amount of nucleostemin mRNA or polypeptide in a cell as compared to a control, such as, but not limited to, at least a 30%, 50%, 75%, 100%, or 200% increase of nucleostemin MRNA or polypeptide. In another example, expression of nucleostemin is decreased as compared to a control. Decreased expression includes, but is not limited to, at least a 20% decrease in the amount of nucleostemin MRNA or polypeptide in a cell as compared to a control, such as, but not limited to, at least a 30%, 50%, 75%, 100%, or 200% decrease of nucleostemin RNA or polypeptide in the cell. Suitable controls include a cell not contacted with an agent that alters nucleostemin expression, such as a wild-type cell, a stem cell, or an untreated tumor cell. Suitable controls also include standard values.

In yet another embodiment, the method includes altering the level of nucleostemin bound to p53. For example, the method can alter the amount of p53 bound to nucleostemin by at least a 20%, such as, but not limited to, at least a 30%, 50%, 75%, 100%, or 200% change in the amount of nucleostemin bound to p53. Suitable controls include the amount of nucleostemin bound to p53 in a cell not contacted with an agent that alters nucleostemin expression, such as a wild-type cell, a stem cell, or an untreated tumor cell.

In a specific, non-limiting example, the nucleostemin has an amino acid sequence set forth as:

(SEQ ID NO: 10)

MKRPKLKKASKRMTCHKRYKIQKKVREHHRKLRKEAKKJ₁GHKKPJ₂KDP
GVPNSAPFKEALLREAELRKQJ₃LEELKQQQKLDRQKEJ₄EXKRKLEXXP
XXXXSNVEXXEXXXUUXXXXXKAKXGKQNXKKLXCQELKKVIEASDXVLE
VLDARDPLGCRCPQXEEAXXQSGXKKLXLXLNKSDLVPKENLEXWLXYLX
KELPTVVFXASTXXKUXXKUTXXXKXKUUXXPFXSXXCXGKEXLWKLLGX
FQXXCXKXXXVGVXGFPNVGKSSXINSLKQEXXCXVGXSMGLTRSMQXVP
LDKQITIIDSPXXIXSPXNSXXALALRSPASIEXXXPXEAASAILSQADX
XQVVLKYTVPXYXXSLXFFTXLAQRRGXHQKGGXPNVEXAKLXWSEWTGA
SLXYYCHPPXSWXXXXXFNEXIXXXMKXGFNLEELEKNNAXSIXXXKGPH
LXNXILFXSSGLTNGIXXEKDIXEEXPXXXEXXQXXUUUUUUXDQEXXXX
EXXXXXSXXXXXEETXEXXXXXXTAXXXSXXSXXLDXXXXEDXXYDFXTD
YX.

J₁ equals R or Q; J₂ equals K or R; J₃ equals Q or R; J₄ equals Q or L; X equals any amino acid; and U equals any amino acid or no amino acid.

In one specific, non-limiting example, the nucleostemin includes an amino acid sequence at least 80% identical to SEQ ID NO: 6. In one example, the polypeptide includes a sequence set forth as SEQ ID NO: 6:

MKRPKLKKASKRMTCHKRYKIQKKVREHHRKLRKEAKKQGHKKPRKDPGV
PNSAPFKEALLREAELRKQRLEELKQQQKLDRQKELEKKRKLETNPDIKP
SNVEPMEKEFGLCKTENKAKSGKQNSKKLYCQELKKVIEASDVVLEVLDA
RDPLGCRCPGVEEATVGSGQKKLVLTLNKSDLVPKENLESWLNYLKKELP
TVVFRASTKPKDKGKTTKRVKAKKNAAPFRSEVCFGKEGLWKLLGGFQET
CSKATRVGVTGFPNVGKSSTTNSLKQEQMCNVGVSMGLTRSMQVVPLDKQ
ITIIDSPSFTVSPLNSSSALALRSPASTEVVKPMEAASAILSQADARQVV
LKYTVPGYRNSLEFFTMLAQRRGMHQKGGIPNVEGAAKLLWSEWTGASLA
YYCHPPTSWTPPPYFNESTVVDMKSGFNLEELEKNNAQSTRATKGPHLAN
SILFQSSGLTNGIIEEKDTHEELPKRKERKQEEREDDKDSDQETVDEEVD
ENSSGMFAAEETGEALSEETTAGEQSTRSFTLDKTTEEDDAYDFSTDYV

Specific, non-limiting examples of nucleostemin polypeptides include polypeptides including an amino acid sequence at least about 80%, 85%, 90%, 95%, or 99% homologous to the amino acid sequence set forth in SEQ ID NO: 6 (see FIG. 1). Specific, non-limiting examples of a nucleostemin polypeptides that are at least about 80% homologous to SEQ ID NO: 6 are SEQ ID NO: 2, SEQ ID NO: 4, and SEQ ID NO: 10.

In a further embodiment, a nucleostemin polypeptide is a conservative variant of SEQ ID NO: 6, such that it includes no more than fifty conservative amino acid substitutions, such as no more than two, no more than five, no more than ten, no more than twenty, or no more than fifty conservative amino acid substitutions in SEQ ED NO: 6. In another embodiment, a nucleostemin polypeptide has an amino acid sequence as set forth as SEQ ID NO: 6.

Specific, non-limiting examples of a nucleostemin polypeptide of use in the methods disclosed herein is a conservative variant of SEQ ID NO: 2 or a conservative variant of SEQ ID NO: 4, as described above. In several embodiments, a conservative variant includes at most one, at most two, at most five, at most ten, or at most fifteen conservative substitutions of the sequence shown in SEQ ID NO: 2 or SEQ ID NO: 4. Generally, a conservative variant will bind to antibodies that immunoreact with a polypeptide including a sequence set forth as SEQ ID NO: 2, and/or will immunoreact with a polypeptide including a sequence set forth as SEQ ID NO: 4.

In the methods disclosed herein, nucleostemin can either be increased or decreased in a cell to inhibit proliferation of the cell or to induce differentiation of the cell. In one embodiment, a nucleostemin polypeptide is administered to the cell of interest. In another embodiment, the activity of a nucleostemin polypeptide is inhibited. In another embodiment, expression of a nucleic acid encoding a nucleostemin polypeptide is induced. In a further embodiment, expression of a nucleic acid encoding nucleostemin is decreased.

Differentiation can be induced, or proliferation decreased, of any cell, either in vivo or in vitro, using the methods disclosed herein. In one embodiment, the cell is a stem cell, such as, but not limited to, an embryonic stem cell, a neuronal progenitor cell, a hematopoietic stem cell, or a pancreatic endocrine progenitor cell.

In one embodiment, the cell is a tumor cell, including a cell of a benign or a malignant tumor (e.g. a cancer cell). Cancer cells include, but are not limited to, tumors of the breast, intestine, liver, lung, ovary, testes, bone, lymphocytes, bladder, skin, prostate, brain, kidney, endocrine system, thyroid, or any other tissue of interest.

In one specific, non-limiting example expression of nucleostemin is increased or decreased to differentiate retinoblastoma cells. Specifically, alteration of nucleostemin is of use in cells in which the retinoblastoma gene (RB) is not itself impaired, such as when the effective impairment of the RB checkpoint is the result of a failure to control RB through p53. Thus, in one specific non-limiting example, a recombinant retrovirus can be constructed to facilitate expression of nucleostenin, and infectivity of retinoblastoma cells is enhanced by derivatizing the env protein or the retrovirus with antibodies specific for retinoblastoma cells, e.g. antibodies to retinal S-antigen (Doroso et al., Invest Opthalizol Vis Sci 26:560-572, 1985; Liao et al. Eur J Immunol 11:450-454, 1981; U.S. Pat. No. 4,444,744).

In yet another embodiment, expression of nucleostemin is increased or decreased in a sarcoma, e.g. an osteosarcoma or Kaposi's sarcoma. In a specific, non-limiting example, a nucleic acid encoding nucleostemin is provided in a viral vector and delivered by way of a viral particle which has been derivatized with antibodies immunoselective for an osteosarcoma cell (see, for example, U.S. Pat. No. 4,564,517 and U.S. Pat. No. 4,444,744; and Singh et al., Cancer Res 36:4130-4136, 1976).

In a still further embodiment, expression of a nucleostemin is altered (increased or decreased) in tissue which is characterized by unwanted de-differentiation and which may also be undergoing unwanted apoptosis. For instance, many neurological disorders are associated with degeneration of discrete populations of neuronal elements. For example, Alzheimer's disease is associated with deficits in several neurotransmitter systems, both those that project to the neocortex and those that reside with the cortex.

Altering the expression or activity of nucleostemin can also be used to inhibit proliferation of smooth muscle cells, and can therefore be used as part of a therapeutic regimen in the treatment of a patient suffering from a condition which is characterized by excessive smooth muscle proliferation. The arterial wall is a complex multicellular structure and is important in the regulation of inflammation, coagulation, and regional blood flow. Vascular smooth muscle cells (SMCs) are located predominantly in the arterial tunica media and are important regulators of vascular tone and blood pressure. These cells are normally maintained in a nonproliferative state in vivo. Arterial injury results in the migration of SMCs into the intimal layer of the arterial wall, where they proliferate and synthesize extracellular matrix components.

Arterial intimal thickening after injury is the result of the following series of events: (1) initiation of smooth muscle cell proliferation within hours of injury, (2) SMC migration to the intima, and (3) further SMC proliferation in the intima with deposition of matrix. The overall disease process can be termed a hyperproliferative vascular disease because of the etiology of the disease process. This process can be biologically induced (as in atherosclerosis, transplant atheroscelerosis) or mechanically induced (as in balloon angioplasty). Thus, a method is provided herein of altering smooth muscle cell proliferation by altering the expression of a nucleostemin.

The level of nucleostemin in a cell can be altered by administration of a nucleostemin polypeptide. For example, nucleostemin can be administered using liposomes, or any other method known to be effective in delivering proteins known to one of skill in the art.

Expression of nucleostemin can be altered by administering a nucleic acid encoding nucleostemin to the cell. In vitro methods for delivery of a nucleic acid are disclosed above. In vivo, expression constructs including a nucleic acid encoding nucleostemin can be administered in any biologically effective carrier, e.g. any formulation or composition capable of effectively transfecting cells in vivo. Approaches include insertion of a nucleic acid encoding nucleostemin in viral vectors including recombinant retroviruses, adenovirus, adeno-associated virus, and herpes simplex virus-1, or recombinant bacterial or eukaryotic plasmids. Viral vectors can be used to transfect cells directly; plasmid DNA can be delivered with the help of, for example, cationic liposomes (lipofectin) or derivatized (e.g. antibody conjugated), poly-lysine conjugates, gramacidin S, artificial viral envelopes or other such intracellular carriers, as well as direct injection of the gene construct or CaPO.sub.4 precipitation carried out in vivo. The particular delivery system of use will depend on such factors as the phenotype of the intended target and the route of administration, e.g. locally or systemically.

In one embodiment, a viral vector containing nucleic acid, e.g., a cDNA, encoding nucleostemnin is utilized. These vectors include, but are not limited, to retroviruses or adenoviruses. A major prerequisite for the use of retroviruses is to ensure the safety of their use, particularly with regard to the possibility of the spread of wild-type virus in the cell population. The development of specialized cell lines (termed “packaging cells”) which produce only replication-defective retroviruses has increased the utility of retroviruses, and defective retroviruses are well characterized for use in gene transfer (for a review see Miller, Blood 76:271, 1990). Thus, recombinant retrovirus can be constructed in which part of the retroviral coding sequence (gag, pol, env) has been replaced by nucleic acid encoding a nucleostemin, rendering the retrovirus replication defective. The replication defective retrovirus is then packaged into virions which can be used to infect a target cell through the use of a helper virus by standard techniques. Protocols for producing recombinant retroviruses and for infecting cells in vitro or in vivo with such viruses can be found in Current Protocols in Molecular Biology, Ausubel, F. M. et al. (eds.), Greene Publishing Associates, Sections 9.10-9.14, 1989. Exemplary retroviruses include pLJ, pZIP, pWE and pEM, which are of use in transfecting neural cells, epithelial cells, endothelial cells, lymphocytes, myoblasts, hepatocytes, bone marrow cells, in vitro and/or in vivo (see for example Eglitis et al., Science 230:1395-1398, 1985; Danos and Mulligan, Proc. Natl. Acad. Sci. U.S.A. 85:6460-6464; Wilson-et al., Proc. Natl. Acad. Sci. U.S.A. 85:3014-3018, 1988; U.S. Pat. No. 4,868,116; U.S. Pat. No. 4,980,286; WO 89/07136; WO 89/02468; WO 89/05345; and WO 92/07573).

It has been shown that it is possible to limit the infection spectrum of retroviruses and consequently of retroviral-based vectors, by modifying the viral packaging proteins on the surface of the viral particle (see, for example WO 93/25234, WO 94/06920, and WO 94/11524). For instance, strategies for the modification of the infection spectrum of retroviral vectors include coupling antibodies specific for cell surface antigens to the viral env protein (Roux et al. Proc. Natl. Acad. Sci. U.S.A. 86:9079-9083, 1989; Julan et al., J. Gen Virol. 73:3251-3255, 1992). Coupling can be in the form of the chemical cross-linking with a protein or other variety (e.g. lactose to convert the env protein to an asialoglycoprotein), as well as by generating fusion proteins (e.g. single-chain antibody/env fusion proteins). Retroviral gene delivery can be further enhanced by the use of tissue- or cell-specific transcriptional regulatory sequences which control expression of the CCR-gene of the retroviral vector.

Adenovirus-derived vectors are also of use with a nucleic acid encoding nucleostemin. The genome of an adenovirus can be manipulated such that it encodes a gene product of interest, but is inactivate in terms of its ability to replicate in a normal lytic viral life cycle (see, for example, Berkner et al., BioTechniques 6:616, 1988; Rosenfeld et al., Cell 68:143-155, 1992). Suitable adenoviral vectors are derived from the adenovirus strain Ad type 5 dl324 or other strains of adenovirus (e.g., Ad2, Ad3, Ad7 etc.). The adenovirus can be a replication-defective adenoviral vector, such as a virus deleted for all or parts of the viral E1 and E3 genes (see, Graham et al. in Methods in Molecular Biology, E. J. Murray, Ed. (Humana, Clifton, N.J., vol. 7. pp. 109-127, 1991)).

Yet another viral vector system of use is the adeno-associated virus (AAV). Adeno-associated virus is a naturally occurring defective virus that requires another virus, such as an adenovirus or a herpes virus, as a helper virus for efficient replication and a productive life cycle. (For a review see Muzyczka et al., Curr. Topics in Micro. and Immunol. 158:97-129, 1992). Other viral vector systems that are of use include herpes virus, vaccinia virus, and other RNA viruses, such as lentiviruses.

In addition to viral transfer methods, non-viral methods can also be employed. Exemplary delivery systems of this type include liposomal derived systems, poly-lysine conjugates, and artificial viral envelopes.

In one specific, non-limiting example, a nucleic acid encoding nucleostemin can be delivered to a cell of interest using liposomes bearing positive charges on their surface (e.g., lipofectins). These liposomes can be tagged with antibodies against cell surface antigens of the target tissue (e.g. see WO 91/06309; Japanese Patent Application 1047381; and European Patent Publication EP-A-43075). In another specific, non-limiting example, the delivery system includes an antibody or cell surface ligand which is cross-linked with a nucleic acid binding agent such as poly-lysine (see, for example, WO 93/04701, WO 92/22635, WO 92/20316, WO 92/19749 and WO 92/06180).

Expression of nucleostemin can be altered by administering an antisense molecule or a ribozyme that specifically binds nucleostemin, or by administering small inhibitory RNA molecules (siRNA).

Antisense molecules are oligonucleotide probes or their derivatives which specifically hybridize (e.g. bind) under cellular conditions, with the cellular mRNA and/or genomic DNA encoding a nucleostemin, so as to inhibit expression of that protein, e.g. by inhibiting transcription and/or translation. The binding may be by conventional base pair complementarity, or, for example, in the case of binding to DNA duplexes, through specific interactions in the major groove of the double helix.

Antisense nucleic acids, namely DNA or RNA molecules that are complementary to at least a portion of a nucleostemin nucleic acid can be used in the methods disclosed herein (e.g. see Weintraub, Scientific American 262:40, 1990). In one specific example, in the cell, the antisense nucleic acids hybridize to the corresponding mRNA, forming a double-stranded molecule. The antisense nucleic acids interfere with the translation of the mRNA, since the cell will not translate a mRNA that is double-stranded. Antisense oligomers of about 15 nucleotides are of use, since they are easily synthesized and are less likely to cause problems than larger molecules when introduced into the target nucleostemin-producing cell. The use of antisense methods to inhibit the in vitro translation of genes is well known in the art (Marcus-Sakura, Anal. Biochem. 172:289, 1988).

Use of an oligonucleotide to stall transcription is known as the triplex strategy since the oligomer winds around double-helical DNA, forming a three-strand helix. Therefore, these triplex compounds can be designed to recognize a unique site on a chosen gene (Maher et al., Antisense Res. and Dev. 1:227, 1991; Helene, Anticancer Drug Design 6:569, 1991). This strategy can be used to produce oligonucleotides that specifically inhibit transcription of a nucleostemin RNA.

Ribozymes are RNA molecules possessing the ability to specifically cleave other single-stranded RNA in a manner analogous to DNA restriction endonucleases. Through the modification of nucleotide sequences which encode these RNAs, it is possible to engineer molecules that recognize specific nucleotide sequences in an RNA molecule and cleave it (Cech, J. Amer. Med. Ass. 260:3030, 1988). A major advantage of this approach is that, because they are sequence-specific, only mRNAs with particular sequences are inactivated.

There are two basic types of ribozymes namely, tetrahymena-type (Hasselhoff, Nature 334:585, 1988) and “hammerhead”-type. Tetrahymena-type ribozymes recognize sequences which are four bases in length, while hammerhead-type ribozymes recognize base sequences 11-18 bases in length. The longer the recognition sequence, the greater the likelihood that the sequence will occur exclusively in the target mRNA species. Either type of ribozyme is of use in inhibiting expression of a nucleostemin.

The present disclosure further provides a method for treating mammalian cells by interfering or inhibiting expression of a nucleostemin in the cells, by exposing the animal cells to an effective amount of an RNA (siRNA) suitable for interfering or inhibiting expression of nucleostemin. The RNA comprises double stranded RNA of about 15 to about 40 nucleotides containing a 0-nucleotide to 5-nucleotide 3′ and/or 5′ overhang on each strand, wherein the sequence of the RNA is substantially identical to a portion of a mRNA or transcript of the nucleostemin. The siRNA can be used to inhibit nucleostemin suitable, either in vivo and in vitro.

The inhibitory RNAs can have unmodified or modified-backbones and/or component nucleosides. Such modifications include, but are not limited to, -thio, 2′-fluro 2′-amino, 2′-deoxy, 4-thio, 5-bromo, 5-iodo and 5-(3-aminoallyl) derivatives of ribonucleosides.

The siRNA can be delivered directly, derived from a viral RNA, or produced from a transgene (Hamilton et al., Science 286:950, 1999; Zamore et al., Cell 101:25, 2000; Hammond et al., Nature 404:293, 2000; Yang et al., Curr. Biol. 10:1191, 2000; Parrish et al., Mol. Cell 6:1077, 2000; Elbashir et al., Genes Dev. 15:188, 2001).

An antisense or small inhibitory RNA construct can be delivered, for example, as an expression plasmid which, when transcribed in the cell, produces RNA which is complementary to at least a unique portion of the cellular mRNA. Alternatively, the antisense construct is an oligonucleotide probe which is generated ex vivo and which, when introduced into the cell causes inhibition of expression by hybridizing with the mRNA and/or genomic sequences encoding one of the subject CCR proteins. Such oligonucleotide probes are preferably modified oligonucleotide which are resistant to endogenous nucleases, e.g. exonucleases and/or endonucleases, and are therefore stable in vivo. Exemplary nucleic acid molecules for use as antisense oligonucleotides are phosphoramidate, phosphothioate and methylphosphonate analogs of DNA (see also U.S. Pat. No. 5,176,996; U.S. Pat. No. 5,264,564; and U.S. Pat. No. 5,256,775). Additionally, general approaches to constructing oligomers useful in antisense therapy have been reviewed, for example, by van der krol et al., Biotechniques 6:958-976, 1988; and Stein et al., Cancer Res 48:2659-2668, 1988.

### Pharmaceutical Preparations and Therapy

In one embodiment, a method is provided for decreasing proliferation of a cell in a subject, or for inducing differentiation of a cell in a subject, including administering a therapeutically effective amount of an agent that alters the level of a nucleostemin polypeptide, and a pharmaceutically acceptable carrier. The nucleostemin polypeptide can be a polypeptide including an amino acid sequence at least 80% identical to SEQ D NO: 6. “Administering” the pharmaceutical composition can be accomplished by any means known to one of skill in the art.

The pharmaceutical compositions are preferably prepared and administered in dose units. Solid dose units are tablets, capsules and suppositories. For treatment of a subject, such as but not limited to a human subject, and depending on activity of the compound, manner of administration, nature and severity of the disorder, age and body weight of the patient, different daily doses are necessary. Under certain circumstances, however, higher or lower daily doses may be appropriate. The administration of the daily dose can be carried out both by single administration in the form of an individual dose unit or else several smaller dose units and also by multiple administrations of subdivided doses at specific intervals.

The pharmaceutical compositions can be administered systemically or locally, such as, but not limited to, by injection directly into a tumor. The compositions are in general administered topically, intravenously, intramuscularly, orally, parenterally, or as implants, but even rectal use is possible in principle. Suitable solid or liquid pharmaceutical preparation forms are, for example, granules, powders, tablets, coated tablets, (micro)capsules, suppositories, syrups, emulsions, suspensions, creams, aerosols, drops or injectable solutions in ampule form and also preparations with protracted release of active compounds, in whose preparation excipients and additives and/or auxiliaries such as disintegrants, binders, coating agents, swelling agents, lubricants, flavorings, sweeteners or solubilizers are customarily used as described above. The pharmaceutical compositions are suitable for use in a variety of drug delivery systems. For a brief review of present methods for drug delivery, see Langer, Science 249:527-1533, 1990.

A therapeutically effective dose of an agent that alters the level of a nucleostemin is the quantity of a compound necessary to inhibit, to cure or at least partially arrest the symptoms of the disorder and its complications. Amounts effective for this use will, of course, depend on the severity of the disease and the weight and general state of the patient. Typically, dosages used in vitro may provide useful guidance in the amounts useful for in situ administration of the pharmaceutical composition, and animal models may be used to determine effective dosages for treatment of particular disorders. Various considerations are described, e.g., in Gilman et al., eds., Goodman And Gilman's: The Pharmacological Bases of Therapeutics, 8th ed., Pergamon Press, 1990; and Remington's Pharmaceutical Sciences, 17th ed., Mack Publishing Co., Easton, Pa., 1990, each of which is herein incorporated by reference.

In clinical settings, systems for the introduction of a nucleic acid encoding nucleostemin, or designed to inhibit the expression of nucleostemin, can be introduced into a subject by any of a number of methods. For instance, a pharmaceutical preparation of the nucleic acid delivery system can be introduced systemically, e.g. by intravenous injection, and specific transduction of the target cells occurs predominantly from specificity of transfection provided by the gene delivery vehicle, the cell-type or tissue-type expression due to the transcriptional regulatory sequences controlling expression of the gene, or a combination thereof. In other embodiments, initial delivery of the recombinant gene is more limited with introduction into the animal being quite localized. For example, the gene delivery vehicle can be introduced by catheter (see U.S. Pat. No. 5,328,470) or by stereotactic injection (e.g. Chen et al., PNAS 91:3054-3057, 1994).

Moreover, the pharmaceutical preparation can consist essentially of the nucleic acid system in an acceptable diluent, or can be a slow release matrix in which the nucleic acid delivery vehicle is imbedded. Alternatively, where the complete delivery system can be produced from recombinant cells, e.g. retroviral packages, the pharmaceutical preparation can include one or more cells which produce the gene delivery system. In the case of the latter, methods of introducing the viral packaging cells may be provided by, for example, rechargeable or biodegradable devices. Various slow release polymeric devices have been developed and tested in vivo in recent years for the controlled delivery of drugs, including proteinacious biopharmaceuticals, and can be adapted for release of viral particles through the manipulation of the polymer composition and form. A variety of biocompatible polymers (including hydrogels), including both biodegradable and non-degradable polymers, can be used to form an implant for the sustained release of viral particles by cells implanted at a particular target site. Such embodiments can be used for the delivery of an exogenously purified virus, which has been incorporated in the polymeric device, or for the delivery of viral particles produced by a cell encapsulated in the polymeric device.

By choice of monomer composition or polymerization technique, the amount of water, porosity and consequent permeability characteristics can be controlled. The selection of the shape, size, polymer, and method for implantation can be determined on an individual basis according to the disorder to be treated and the individual patient response. The generation of such implants is generally known in the art (see, for example, Concise Encyclopedia of Medical & Dental Materials, ed. by David Williams (MIT Press: Cambridge, Mass., 1990); U.S. Pat. No. 4,883,666). In another embodiment of an implant, a source of cells producing a recombinant virus is encapsulated in implantable hollow fibers. Such fibers can be pre-spun and subsequently loaded with the viral source (U.S. Pat. No. 4,892,538; U.S. Pat. No. 5,106,627), or can be co-extruded with a polymer which acts to form a polymeric coat about the viral packaging cells U.S. Pat. No. 4,391,909; U.S. Pat. No. 4,353,888). Again, manipulation of the polymer can be carried out to provide for optimal release of viral particles.

### Screening for Agents that Affect Differentiation, Proliferation, and/or Senescence of a Cell

A method for screening for agents that affect differentiation, proliferation, and/or senescence is provided herein. Nucleostemin interacts with p53 (see the examples section below). Thus, in one embodiment, the method to identify an agent of interest includes contacting p53 and a nucleostemin polypeptide including an amino acid sequence at least 80% identical to SEQ ID NO: 6 with an agent of interest in vitro. The binding of p53 and the nucleostemin polypeptide is then evaluated. A decrease in the binding of p53 and the nucleostemin polypeptide indicates that the agent affects differentiation, proliferation, and/or senescence of the cell. Suitable controls include the binding of p53 and nucleostemin in the absence of any agent or in the presence of a carrier, such as a buffer. A suitable control also includes the binding of p53 and nucleostemin in the presence of an agent known to affect this interaction. Suitable controls also include standard values. “Incubating” includes conditions which allow contact between the test compound and p53 and/or nucleostemin. “Contacting” includes in solution and solid phase.

Prior to performing any assays to detect interference with the association of p53 and a nucleostemin, rapid screening assays could be used to screen a large number of agents to determine if they bind to p53 or nucleostemin. Rapid screening assays for detecting binding to HIV proteins have been disclosed, for example, in U.S. Pat. No.5,230,998. In this type of assay, p53 or the nucleostemin is incubated with a first antibody capable of binding to p53 or the nucleostemin, and the agent to be screened. Excess unbound first antibody is washed and removed, and antibody bound to the p53 or nucleostemin is detected by adding a second labeled antibody which binds the first antibody. Excess unbound second antibody is then removed, and the amount of the label is quantitated. The effect of the binding effect is then determined in percentages by the formula:
(quantity of the label in the absence of the drug)−(quantity of the label in the presence of the drug/quantity of the label in the absence of the drug)×100
Agents that are found to have a high binding affinity to the p53 or nucleostemin can then be used in other assays more specifically designed to test inhibition of the p53/nucleostemin interaction.

Examples of agents that interfere with an interaction of p53 and a nucleostemin, identified using such an assay, include: chemical compounds; fragments and fusions of nucleostemin; peptidomimetics; antibodies; synthetic ligands that bind nucleostemin or p53, agents which cause the disassociation of p53 and nucleostemin; appropriate nucleostemin or p53 fragments, or other fragments of the natural or synthetic ligands or chemical compounds which bind to p53 and prevent the interaction of p53 and nucleostemin, and thereby affect cell differentiation, proliferation, and/or senescence. The determination and isolation of ligand/compositions is well described in the art. See, e.g., Lerner, Trends Neuro Sci. 17:142-146, 1994.

The test compound may also be a combinatorial library for screening a plurality of compounds. Compounds identified in the disclosed methods can be further evaluated, detected, cloned, sequenced, and the like, either in solution of after binding to a solid support, by any method usually applied to the detection of a specific DNA sequence, such as PCR, oligomer restriction (Saikl et al., Bio/Technology 3:1008-1012, 1985), allele-specific oligonucleotide (ASO) probe analysis (Conner et al., Proc. Natl. Acad. Sci. U.S.A. 80:278, 1983), oligonucleotide ligation assays (OLAs) (Landegren et al., Science 241:1077, 1988), and the like. Molecular techniques for DNA analysis have been reviewed (Landegren et al., Science 242:229-237, 1988).

Binding can be measured by any means known to one of skill in the art. For example competitive binding assays can be utilized. In another example, a nucleostemin, such as a polypeptide including an amino acid sequence at least 80% identical to SEQ ID NO: 6, is attached to a matrix, or introduced into wells of a microtiter plate. Extracts that contain normal or modified forms of p53 are incubated with the matrices or plates, and the p53 protein adsorbs onto the nucleostemin but not onto control matrices or wells that lack nucleostemin. After washing away the unabsorbed p53, the matrices or plates are analyzed by standard methods such as ELISA for detection of the adsorbed p53.

Drug candidates are added to the assay wells to determine whether any agent, such as a chemical compound, antibody or peptide, blocks binding of p53 to the matrices or plates that contain the nucleostemin. The assays could also be done inversely, by binding p53 and by studying the adsorption of nucleostemin onto the p53. Such assays can also be performed with small fragments of p53 that contain only the domain needed for nucleostemin binding.

The disclosure is further illustrated by the following non-limiting Examples.

### EXAMPLES

### Example 1

### Materials and Methods

Cortical stem cell culture and transfection protocol: Primary cortical stem cell culture was prepared from rat cerebral cortices of E14.5 (Johe et al., Genes Dev 10:3129-3140, 1996; Tsai et al., J. Neurosci. 20:3725-3735, 2000). Cells were expanded in the presence of bFGF (20 ng/ml) for 4 days and were replated at 2×10⁵ per 10 cm plate for 4 days before induction of differentiation. Neurospheres were derived from the subventricular zone of the lateral ventricles of adult rats and cultured in suspension. Transfection was carried out on cells grown as monolayers on glass coverslips using LipofectAmine Plus reagent (Invitrogen).

Molecular cloning and RT-PCR: For PCR-select cDNA subtraction (Clontech), polyA⁺ RNA (2 μg) was used in the cDNA synthesis process. Due to the nature of the screening methodology, genes that are part of a family with close homology or of low abundance may be missed. The complete cDNAs for murine (muNS) were isolated from a rapid-screen cDNA library panel derived from E12.5 Swiss Webster mice (OriGene Technologies) and the rat nucleostemin (raNS, Sprague-Dawley) from RACE PCR on 1^{st} stranded cDNA derived from cortical stem cells. Semi-quantitative PCR reactions were carried out in 25 μl with 1 μl of the first strand cDNA solution and 0.4 μM of each primer. The linear range was predetermined by pilot experiments.

Antibody synthesis and immunocytohistochemistry: 2438 antibody was raised in chicken (Aves Labs) and 1164 antibody in rabbit (Biosynthesis, Inc.). The 2438 antibody recognized primarily rat and murine nucleostemin and the 1164 antibody recognized human, mouse, and rat proteins. Primary antibodies and their dilution used in this study were: chicken (2438, 1:500) and rabbit (1164, 1:1000) α nucleostemin; rat α-BrdU (Harlan SeraLab), 1:200; α-HA (mouse or rabbit, Covance), 1:1000; α nucleolin (RDI, 1:1000); α-phospho-Histone H3 (Upstate), 1:400; α-activated Caspase3 (CM1, 1:5000). Secondary antibodies were Rhodamine Red or FITC-conjugated (Jackson). Cells were fixed with ice-cold methanol (for α-nucleostemin and nucleolin) or 4% paraformaldehyde. For immunohistochemistry, embryos were harvested from time-pregnant CD1 mice and embedded in OCT without fixation. Frozen sections (16 μm) were fixed in 4% paraformaldehyde for 2 minutes and incubated with 1164 (1:1500). Immunohistochemistry was performed with ABC kit (Vector) and 3,3′-diaminobenzidine (DAB) color reaction (SigmaFast). For BrdU double-labeled immunocytochemistry, a 15-minute pulse of BrdU (40 μM) was administered to mark S-phase cells for an 18-hour pulse of BrdU (10 μM) was used to label all cells in cell cycle. After carrying out immunostaining of the first antibody, cells were fixed, treated with 4N HCl for 10 minutes to denature the DNA, and continued with α-BrdU staining.

siRNA knockdown experiment: Cells were incubated with siRNA (100 nM, Dharmacon Research, CO) complexed with oligofectamine Invitrogen) for 18 hours. BrdU labeling (40 μM for 15-minutes pulse or 10 μm for 18-hour pulse) was measured 72 hours after transfection. The sequences for siRNA are: NS-specific siRNA: AAGAACUAAAACAGCAGCAGAdTdT (SEQ ID NO: 7); control siRNA; AACAUUCAGACUGGGAAAUGGdTdT (SEQ ID NO: 8) for rat cells and AAUCAGACGUGGACCAGAAGAdTdT (SEQ ID NO: 9) for human cells.

GST binding, coimmunoprecipitation and Western blot analysis: 293 cells (100 mm dish) were transfected with 5-10 μg of DNA plasmid and harvested 36 hours later. Cells were lysed in extraction buffer (PBS/1% Triton X-100/1 mM PMSF/protease inhibitors) and assayed for protein concentration (Bradfort colorimetric assay, Bio-Rad). Lysates (500 μg) were incubated with (1) immobilized purified GST or GST fusion protein, and glutathione agarose in 1 ml volume for GST pull-down experiment, or (2) antibody (1 μg for purified antibody or 10 μl for crude serum) and protein A-sepharose (50 μl) for coimmunoprecipitation for 2 hours at 4° C. Beads were washed with PBS once, PBS/500 mM NaCl twice, and PBS twice. The antibody used in Western blot detection are: nucleostemin (2438 and1164, 1:1000); p53 (FL393, 1:500, Santa Cruz; Ab7, 1:2000, Calbiochem). The antibody used in developmental Western blot detection are: nucleostemin (2438 and1164, 1:1000); B23 (C-19, 1:200, Santa Cruz); PCNA (1:500, Novocastra).

Flow cytometry: To enrich hematopoietic stem cells, bone marrow cells were harvested from adult C57BL/6J mice, depleted of cells with lineage-specific markers (CD4, CD8α, B220, Gr-1, and Mac-1) by magnetic sorting, incubated with FITC-conjugated α-c-kit antibody, and sorted on a FACS Vantage II flow cytometer (Beckman Coulter). For B lymphocyte and granulocytes lineages, bone marrow cells were depleted of the other hematopoietic lineages and then positively sorted with B220 or Gr-1 antibody respectively by flow cytometry.

### Example 2

### Cloning of Nucleostemin

Clonal analysis shows that CNS stem cells can be isolated from the developing rat brain and preferentially enriched in basic fibroblast growth factor (bFGF)-expanded culture (Johe et al., Genes Dev. 10-3129-3140, 1996). Before differentiation, all stem cells divide and express nestin, an intermediate filament characteristic of neuroepithelial precursors (Lendahl et al., Cell 60:585-595, 1990). After withdrawal of bFGF and addition of 10% fetal bovine serum (FBS) for 8 days (d), these cells differentiate efficiently into neurons, astrocytes and oligodendrocytes (FIG. 1A). Taking advantage of the rapid differentiation of CNS stem cells in vitro, a subtractive library was constructed using the undifferentiated pool as tester and the differentiated pool as driver. From this library, two novel clones (SR204 and SR75) and a known gene, 3-hydroxy-3-methylglutaryl coenzyme A reductase (HMG-CoA R), were identified as highly enriched in cortical stem cells. HMG-CoA R, a rate-limiting enzyme in cholesterol biosynthesis, has been shown to have a cholesterol-independent role in regulating primordial germ cell migration in Drosophila (Van Doren et al., Nature 396:466-469, 1998). Disclosed herein is information on one of the two novel clones SR204 found to be expressed abundantly in the stem cell population and rapidly downregulated during differentiation (FIG. 1B).

SR204, named nucleostemin because of its nucleolar localization and preferential expression in cortical stem cells, hybridized to a 1.8-kb transcript. The complete cDNA clones comprised 1810 nt for rats (raNS, SEQ ID NO: 1) and 1813 nt for mice (muNS, SEQ ID NO: 1).

Sequence analysis revealed an open reading frame (ORF) of 1617 nt, an in-frame stop codon in the 5′ untranslated region (UTR), an optimal Kozak sequence, and polyadenylation signals in the 3′ UTR, representing the full-length mRNAs. The ORF encoded a 538 amino acid protein of 61 kDa (pI, 9.4). The raNS (SEQ ID NO: 2) and muNS (SEQ ID NO: 4) shared 90% identity and 94% similarity. PROSITE analysis and Conserved Domain search identified two consensus motifs that define GTP-binding proteins (the G4 motif KXDL, codons 177-180; the G1 or P-loop GXXXXGK(S/T), codon 256-263), a highly basic region in the amino terminus (codons 2-46; pI, 12.4) and an acidic domain in the carboxyl end (codon 460-503; pI, 3.7). PSORT analysis revealed multiple nuclear localization sequences and a coiled-coil domain (FIGS. 1C, 1D). A BLAST search in the GenBank database identified a human homologue (Accession No. AAH01024) that shares 81% identity with the rat sequence. The human gene was isolated based on its estrogen-induced increased expression in breast cancer cell lines.

### Example 3

### Nucleostemin is a Nucleolar Protein

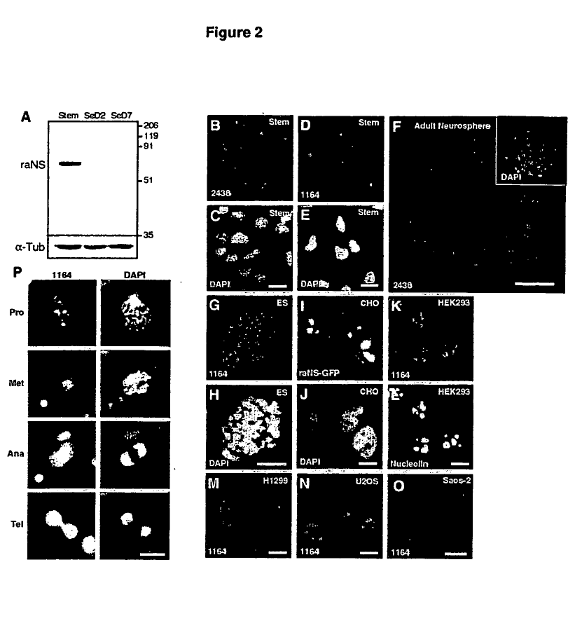

To define the intracellular distribution of nucleostemin protein, a chicken polyclonal antibody (2438) was raised against a peptide corresponding to murine nucleostemin codon 522-538 (DRASQEDETYDFTTDYI, amino acids 522-538 of SEQ ID NO: 4. Western analysis detected a 60-70 kD band of strong intensity in cortical stem cell lysates but only weakly in the 2d and 7d serum-differentiated cultures (FIG. 2A). Immunofluorescence revealed strong signals in one or several nodular structures within the nucleus of embryonic cortical stem cells (FIGS. 2B, 2C). The specificity of the staining pattern was supported by a second antibody (1164) raised against a different epitope in rat nucleostemin (codon 32-49: LRKEAKKRGHKKPRKDPG (amino acids 3249 of SEQ ID NO: 2). (FIGS. 2D, 2E).

Nodular staining with nucleostemin antibody was also seen in cells derived from adult subventricular zone expanded as neurospheres (Reynolds et al., Science 255:1707-1710, 1992) (FIG. 2F) and pluripotent ES cells (FIGS. 2G, 2H), and in Chinese hamster ovary (CHO) cells transfected with a construct encoding raNS-eGFP (green fluorescent protein) fusion protein (FIGS. 2I, 2J). These nodular structures are devoid of DAPI staining and contain the known nucleolar protein nucleolin as seen in HEK293 cells (FIGS. 2K, 2L). Additionally, nucleostemin was found in the nucleoli of the three human cancer cell lines examined, including H1299 (a p53-null non-small cell lung cancer, FIG. 2M), U2OS (an osteosarcoma with wild-type p53, FIG. 2N), and Saos-2 (a p53-null osteosarcoma, FIG. 2O). During interphase, nucleostemin was easily seen in the large nuclei of CHO and HEK293 cells where it was predominantly localized in the nucleolus and at a lower level in the nucleoplasm. When cells entered mitosis, the protein dissociated from the nucleolar region. At prophase (Pro), a portion of nucleostemin was still associated with the nucleolus. During metaphase (Met), anaphase (Ana) and telophase (Tel), nucleostemin was diffusely distributed (FIG. 2P).

### Example 4

### The Expression of Nucleostemin During CNS Development

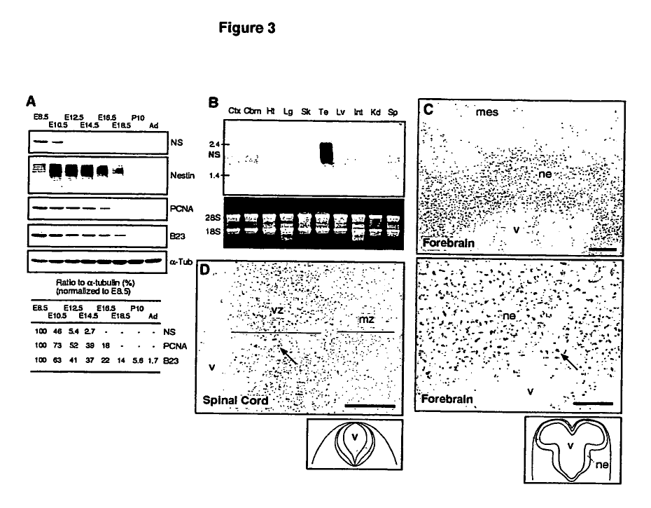

Western blot analysis shows that the expression of nestin peaked around E10.5-E14.5. The nucleostemin protein was elevated as early as E8.5 and declined considerably after E10.5 in the mouse cerebral cortex (FIG. 3A). In contrast, a proliferative marker PCNA and a nucleolar protein B23 decreased gradually and proportionally from E8.5 until birth, reflecting the increase in cells exiting cell cycle. These results are consistent with the notion that nucleostemin is preferentially expressed in vivo by neuroepithelial precursors at a stage before the peak of nestin expression and the precisely timed terminal division of neurons that correlates with the phase of PCNA and B23 expression. Northern analysis of adult rat tissues shows that the mRNA of nucleostemin is present at high levels only in the adult testis (FIG. 3B).

In E10.5 mouse embryos, nucleostemin staining was seen in the neuroepithelial cells in the forebrain (FIG. 3C), midbrain, hindbrain and spinal cord (FIG. 3D). While the E10.5 forebrain contains mainly neural precursors and few differentiated cells, many neurons have already differentiated in the spinal cord at this stage. Consistent with the different onset of differentiation along the CNS axis, nucleostemin signals were strongest in the forebrain compared to the spinal cord at E10.5. Within the spinal cord, the proliferating cells in the ventricular zone expressed nucleostemin at higher levels than the mantle zone where differentiating neurons reside (FIG. 3D). These results show that nucleostemin is highly expressed by early stage CNS precursors in vivo.

### Example 5

### Nucleostemin expression is downregulated When Stem Cells Differentiate into Dividing Progenitors and Prior to Cell-Cycle Exit

The Western blot data described herein indicate that the expression of nucleostemin is switched off while PCNA and B23 expression is maintained (FIG. 3A), suggesting that cells continue to proliferate for a period of time after nucleostemin expression is lost. In culture, nucleostemin was found in almost all rat embryonic cortical stem cells (FIG. 4A), but became undetectable after treatment with ciliary neurotrophic factor (CNTF) for 8 days where 98% of the cells turn into astrocytes (FIGS. 4B, 4C) (Johe et al. 1996). To examine if nucleostemin expression is turned off at the stem cell-to-dividing progenitor or the dividing progenitor-to-postmitotic progeny transition, the expression of nucleostemin was analyzed in 2 day and 8 day CNTF-differentiated cultures labeled with a 15-minute pulse of bromodeoxyuridine (BrdU). If nucleostemin reflects the actual state of proliferation and is turned off when progenitors become postmitotic, the level of nucleostemin should be higher in the S-phase cells labeled with BrdU. After 2 days and 8 days of differentiation in CNTF, the expression level of nucleostemnin was significantly reduced in both the dividing and non-dividing cells (FIG. 4D, CNTF/D2, CNTF/D8), indicating that this down-regulation occurs as an early step in the differentiation of proliferating glial precursors. Such an abrupt change of expression is not restricted to the glial lineage. When cortical stem cells differentiated into neurons, astrocytes and oligodendrocytes in 10% FBS, the expression of nucleostemin was also rapidly attenuated in all cells (FIG. 4D, SeD2, SeD8). These results show that both the dividing cells and the terminally differentiated cells in all lineages present in the 8 day-differentiated cultures were nucleostemin-negative. Thus nucleostemin expression is not simply a reflection of the proliferative state but is characteristic of an early, multipotential state.

The nucleostemin expression level in adult bone marrow cells was compared by Western analysis (FIG. 4E). Consistently with the findings in the nervous system, nucleostemin protein could be detected in the c-kit⁺/lin⁻ population that is enriched for adult hematopoietic stem cells, but not in the lineage-committed B-lymphocytes (B220-positive) or granulocytes (Gr-1-positive). The sustained expression of PCNA suggests that the decrease in nucleostemin expression occurs in cells that are still dividing. These results strengthen the view that nucleostemin is expressed in uncommitted cells and the level of expression does not simply reflect the number of dividing cells.

### Example 6

### Roles of Nucleostemin in Maintaining the Proliferation of Cortical Stem Cells and U2OS Cancer Cell Line

To establish the functional importance of nucleostemin, cortical stem cells were transfected with either control or nucleostemin (NS)-specific small RNAi (siRNA), AAGAACUAAAACAGCAGCAGAdTdT (SEQ ID NO: 7), for 18 hours and assayed for BrdU labeling 72 hours later (Elbashir et al., Nature 411:494-498, 2001). As shown in FIG. 5A (left panel), cultures transfected with NS-specific siRNA consistently showed a higher percentage of non-cycling cells, represented by non-labeled cells after an 18-hour BrdU pulse, than the control group (16.5%±1.4% versus 6.9%±0.5%, mean+standard error mean (s.e.m.), p<0.001). Immunostaming revealed a consistent decrease in the nucleostemin-staining intensity in cells treated with NS-specific siRNA compared to the control-siRNA treated cells (right panel). To obtain a better transfection efficiency, siRNA knockdown experiments were conducted in U2OS cells (FIG. 5B). Western analysis showed an 80% reduction in nucleostemin protein in cultures treated with NS-specific siRNA (FIG. 5B, left panel, Exp) as compared with cultures transfected with control siRNA (Cntrl) 2 days after transfection. Additionally, only in NS-specific siRNA-treated culture were interphase-phase cells seen with diminished nucleostemin staining (FIG. 5B, right panel, indicated by arrows). The percentage of cells in S-phase of these nucleostemin-negative cells was considerably less than that of control-siRNA transfected cultures (8.3%±2.1% versus 48.6%±2.1%, mean±s.e.m., p<0.001, middle panel). These experiments demonstrate that knockdown of nucleostemin protein results in a loss-of-function phenotype as evidenced by a decrease in proliferation and support a role for nucleostemin in maintaining the cell division of CNS stem cells and U2OS cells.

### Example 7

### Overexpression of Nucleostemin Elicits Loss-of-Function Phenotypes in Embryonic Cortical Stem Cells

To gain more insight into the function of nucleostemin, the gene dosage was increased in cortical stem cells and the consequences were analyzed (FIGS. 5C, 5D). Exogenous nucleostemin was introduced by transfecting stem cells with the plasmid encoding hemagglutinin (HA)-tagged raNS under the control of a constitutively active EF-1α promoter (Mizushima et al., Nucleic Acids Res. 18:5322, 1990). The percentage of non-cycling cells (FIG. 5C, right panel, indicated by arrows) was measured in two different paradigms: an undifferentiated culture where stem cells were maintained in bFGF (FIG. 5C bar graph, left), and a differentiating culture where stem cells were differentiated into astrocytes by 2d CNTF treatment (FIG. 5C bar graph, right). In both conditions, more of the raNS-overexpressing cells became non-mitotic than the control-transfected cells (13.6%±1.4% versus 5.0%±7% in bFGF culture; 77.2%±3.4% versus 34.2%±2.7% in 2d CNTF culture, mean±s.e.m., p<0.001). To estimate the number of cells in mitosis, anti-phospho-Histone H3 (α-p-H3) staining was used (FIG. 5D, right panel). Only cells in prophase were included in this analysis because the nucleolar structures disperse in later stages of mitosis, which made it impossible to determine if those cells were expressing low-level of exogenous protein. In accord with the increase in non-dividing cells, the population overexpressing nucleostemin contained less cells in prophase than the control-transfected population (3.0%±0.3% versus 5.2%±0.3%, meann±s.e.m., p<0.001) (FIG. 5D, left panel. These results show that the misexpression of nucleostemin at a high level causes cells to exit cell cycle, a loss-of-function phenotype similar to the knockdown experiment. This is consistent with previous studies showing that elevated levels of gene dosage in a complex pathway may perturb the normal development and/or function of cells overexpressing the gene (Hay et al., Proc. Natl. Acad. Sci. U.S.A. 94:5195-5200, 1997); Yang et al., Nat. Genet. 22:327-335, 1999).

### Example 8

### Functional Importance of the Basic and GTP-Binding Domains in Nucleostemin

To illuminate how excessive nucleostemin blocks cell-cycle progression and to further dissect the molecular mechanism of nucleostemin function, a series of deletion analyses were conducted. Constructs with engineered C-terminal GFP-fusion proteins were generated with deletion of the basic domain (dB, codons 2-46), the coiled-coil domain (dCC, codons 61-87), the G4 GTP-binding motif (dG4, codons 177-180), the G1 GTP-binding motif (dG1, codons 256-263), the acidic domain (dA, codons 460-503), or both the basic and GI domains (dB/G1) (FIG. 6A, left panel and FIGS. 1C, 1D). When introduced into U2OS cells, all mutants were localized in the nucleus but displayed different patterns of distribution within the nucleus (FIG. 6A, right panel). The nucleolar distribution of nucleostemin depends mostly on the N-terminal basic domain. Without this region, the mutant protein (dB) becomes more diffuse in the nucleoplasm. On the other hand, mutants lacking either G4 or G1-GTP-binding motif (dG4, dG1) form irregular aggregates associated with the nucleolar region labeled by α-nucleolin staining, and distort the nucleolar structure. To test whether the ability of these mutants to form aggregates depends on their nucleolar localization, a double mutant (dB/G1) was made that lacked both the basic and the G1 domain. While this mutant protein still forms aggregates, it is becomes dissociated from the nucleolus. Neither deletion of the coil-coiled domain (dCC) nor the acidic domain (dA) produces a consistent change in the intracellular distribution of nucleostemin or the morphology of nucleoli (FIG. 6A). These results demonstrate that the basic domain is required for the nucleolar localization and that the GTP-binding motifs are required to form appropriate nucleolar structure.

### Example 9

### Overexpression of Nucleostemin, Especially Mutants Lacking GTP-Binding Motif, Disrupts Cell-Cycle Progression and Induce cell Death

In FIG. 5 it is shown that perturbation of nucleostemin expression disrupts cell-cycle progression. To better define this effect, cell-cycle kinetics was analyzed in cells expressing wild-type and mutated nucleostemin. The proportion of cells in prophase was estimated by an antibody that recognizes the phosphorylated form of histone H3 (α-p-H3), and the percentage of S-phase cells was given by the number of BrdU-labeled cells after a 15-minute pulse. Consistent with the previous results in cortical stem cells (FIG. 5D), overexpression of wild-type nucleostemin, as well as dCC mutant, caused a significant decrease in the percentage of cells in prophase (1.8%±1% and 1.8%±0.2% respectively, versus 3.1%±0.4% in control, mean±s.e.m., p<0.01).

In contrast to the wild-type protein, deletion of the N-terminal basic domain had no effect on the number of prophase cells (2.8%±0.1%), suggesting that nucleolar localization was required for the overexpression phenotype seen with the wild-type protein. Expression of mutants lacking the G4 or G1 domains caused a dramatic decrease in the number of cells in prophase (0.0%±0.0% and 0.2%±0.0% respectively). Again, this effect depends partially on the basic domain as overexpression of dB/G1 resulted in a higher number of cells in prophase than the G1 deletion alone (0.2%±0.0% versus 0.8%±0.0%, p<0.05) (FIG. 6B, left panel).

The 15 minute pulse with BrdU allowed measurement of the number of cells in S-phase. Surprisingly, there were no changes in the number of S-phase cells among different mutants, wild-type nucleostemin and control (FIG. 6B, middle panel). However, the BrdU labeling pattern showed a clear difference between cells transfected with the GTP-deleted mutants and the wild-type protein. In the mutants lacking the GTP-binding motifs, the BrdU labeling was restricted to small regions in the nucleus, indicating that chromatin replication was blocked (dG1, dG4, dB/G1, FIG. 6B, right panel). None of the other deletions had this effect. These findings indicate that dysregulation of GTP binding in nucleostemin blocks cell-cycle progression in the late S-phase.

To determine the outcome of cell-cycle arrest in the overexpression experiments, cell death was measured in cells expressing the mutant proteins. Cell death was assayed by measuring the expression of the proteolytically activated form of Caspase3 as an indicator of apoptosis (CM1) (Srinivasan et al., Cell Death Differ 5:1004-1016, 1998). Shown in FIG. 6C (left panel), the percentage of Caspase3⁺ cells increased significantly in all mutants lacking the GTP-binding domains when expressed in U2OS cells. The increase in cell death is partially dependent on the N-terminal basic domain, as the apoptotic cells in the dB/G1 mutant were diminished compared to the dG1 mutant (10.9%±1.3% versus 17.3%±1.3%, p<0.005). The increase in cell death by these mutants also depended on the presence of wild-type p53 as the same mutants expressed in Saos-2 cells, a p53-null osteosarcoma cell line, exhibited no significant changes in the percentage of apoptotic cells from the wild-type protein or control (FIG. 6C, right panel).

### Example 10

### Nucleosteniin and p53 Exist in a Protein Complex

p53 is a key cell-cycle checkpoint protein that is involved in stress response, tumor suppression and premature aging. To elucidate the role of p53 in mediating the overexpression phenotypes of nucleostemin, changes in the expression level of p53 and ARF was first examined in CNS stem cells and differentiated cultures (FIG. 6D). ARF is a tumor suppressor that stabilizes p53 by acting through MDM2 (Kamijo et al., Proc. Natl. Acad. Sci. U.S.A. 95:8292-8297, 1998; Zhang et al., Cell 92:725-734, 1998; Tao et al., Proc. Natl. Acad. Sci. U.S.A. 96:6937-6941, 1999), and, like nucleostemin, is localized in the nucleolus. Using semi-quantitative RT-PCR, it was demonstrated that p53 was present in the stem cell population and its level of expression remained unchanged throughout differentiation. On the other hand, the expression of ARF was low in the CNS stem cells and upregulated as they differentiated, a pattern opposite to that seen with nucleostemin.

Despite its nucleolar distribution, ARF has been shown to physically interact with p53 (Kamijo et al., Proc. Natl. Acad. Sci. U.S.A. 95:8292-8297, 1998). Next, it was demonstrated that nucleostemin, like ARF, could also bind p53. In GST-raNS pull-down experiments, raNS retained specifically the full-length p53 (FIG. 6E, left upper panel) but not a proteolytic product of p53 (lower band in the input lane). This binding was not seen with the negative control that used the backbone GST protein to pull down p53. The interaction between p53 and nucleostemin could be further demonstrated by (1) GST pull-down assay using p53 as the GST fusion protein (left lowerpanel) and (2) coimmunoprecipitation of the endogenous p53 and nucleostemin protein complex by 1164 antibody in non-transfected HEK293 cells (right panel). The specificity of this interaction was emphasized by its requirement for both nucleostemin protein and α-nucleostemin antibody. The presence of p53 in the precipitated endogenous protein complex is believed to show that the association between nucleostemin and p53 occurs under physiological conditions.

### Example 11

### The Basic Region of Nucleostemin Mediates its Interaction With p53

To map the interacting region between nucleostemin and p53, HA-tagged mutant proteins were expressed in H1299 cells and incubated with agarose-bound GST-p53 fusion protein in GST pull-down experiments. The amount of mutant proteins retained by GST-p53 fusion was measured by Western blot and presented as the ratio to the input proteins. As shown in FIG. 6F, deletion of the basic domain (dB) significantly reduced the binding of nucleostemin to p53 (17%), whereas the amount of p53 bound by other mutants either had little change (dG1) or a mild 1.5-fold increase (dG4) or reduction (dCC, dA). This data showed that the interaction of nucleostemin with p53 is mediated through its N-terminal basic domain.

Thus, as disclosed herein, novel nucleolar proteins have been identified, termed nucleostemins. These proteins are expressed by developing CNS stem cells. In addition, nucleostemin is present in adult subventricular zone CNS cells expanded as neurospheres, ES cells, other tissues from developing embryos, and several human cancer cell lines, but absent in most adult cells. In the hematopoietic system, nucleostemin is found in a database of genes enriched in Sca-1⁺, AA4.1⁺, c-kit⁺ fetal liver hematopoietic stem cells (SC|LL2GRID-78|14254) (Phillips et al., Science 288:1635-1640, 2000), and is preferentially expressed in the c-kit⁺/lin⁻ adult bone marrow population that is enriched for hematopoietic stem cells.

The expression of nucleostemin is reduced considerably when the expression of PCNA (a proliferative marker) and B23 (a major nucleolar protein) still remains high during CNS stem cell differentiation both in vitro and in vivo. When cortical stem cells differentiate into neurons, astrocytes, and oligodendrocytes, nucleostemin levels are reduced in both dividing and post-mitotic progeny. In the hematopoietic system, B-lymphocytes and granulocytes are nucleostemin-negative, but they express the same level of PCNA as the stem cell-enriched population. These results demonstrate that nucleostemin is expressed predominantly in primitive but not differentiated cells, and the downregulation of nucleostemin is related to terminal differentiation.

The protein knockdown experiments suggest that nucleostemin functions to maintain the proliferative capacity of stem cells and cancer cells. After siRNA knockdown, only 8% of U2OS cells remain in the cycle. Overexpression of nucleostemin also reduces the proliferative rate of stem cells and U2OS cells. Deletion analysis provides an explanation for the overexpression phenotype as well as new insights into the regulatory control of nucleostemin. The deletion study shows that mutants missing the N-terminal basic region distribute diffusely in the nucleoplasm and mutants lacking the GTP-binding sites accumulate as nucleolar aggregates and disrupt the nucleolar structure. When both the basic region and the G1 GTP-binding motif are deleted, nucleostemin is aggregated outside the nucleolus. These results indicate that the N-terminal basic domain determines the nucleolar localization and the GTP-binding motifs regulate the formation of protein aggregates.

Overexpression of nucleostemin causes a decrease in prophase but not S-phase cells, and an increase in cell death that is dependent on the N-terminal basic domain. The effect of overexpression on the numbers of cells in mitosis and cell survival is particularly augmented in the case of mutants lacking the GTP-binding sites. Although the number of cells in S-phase remains the same, the BrdU-labeling patterns are quite different in cells expressing these GTP-deleted mutants compared to the wild-type protein. The punctuate pattern of BrdU labeling occurs between aggregates of the mutant proteins indicating that DNA replication is initiated but does not progress through the chromatin regions associated with high levels of nucleostemin. In agreement with this finding, the percentage of cells in prophase is under-represented in cells expressing the same mutants, and they also show a dramatic effect on cell survival. Without being bound by theory, these data suggest that excess nucleostemin may directly disrupt the nucleolar complex or indirectly cause a block in cell-cycle progression in late S-phase leading to cell death (FIG. 7).

Small GTP binding proteins utilize the conserved GTP-binding structure as a molecular switch to regulate fundamental cellular functions. Drawing analogy from other small GTPases, one can infer that the dynamic shuttling of nucleostemin between the nucleolar and the nucleoplasmic compartment could be regulated by the GTP-binding state of nucleostemin. Thus, without being bound by theory, nucleostemin is believed to form a complex with other nucleolar proteins when it is in a non-GTP bound form and becomes dissociated upon binding with GTP. Disruption of this regulatory mechanism can result in cell-cycle arrest and cell death. The increased cell death in cells expressing the GTP-deleted mutants is abolished when the same constructs are expressed in p53-null Saos-2 cells, indicating that the cell-cycle arrest and cell death effects are mediated by a p53-dependent pathway. In addition, overexpression phenotypes can be partially rescued by deletion of the N-terminal basic domain, which is required for p53 binding. Thus, without being bound by theory, nucleostemin may permit a GTP-regulated and stem cell-specific control of p53 regulation.

Nucleolar proteins have been shown to regulate cell proliferation and growth by controlling ribosome biosynthesis and p53 functions (Drayton et al., Curr. Opin. Genet. Dev. 12:98-104, 2002; Du et al., Cell 109:935-848, 2002; Michael et al., Curr. Opin. Genet. Dev. 12:53-59, 2002). The activation of p53 has been linked to cell-cycle arrest and apoptosis in response to stress. p53 function can be modulated by several nucleolar proteins including ARF (Kamijo et al., Proc. Natl. Acad. Sci. U.S.A. 95:8292-8297, 1998; Zhang et al., Cell 92:725-734, 1998; Tao et al., Proc. Natl. Acad. Sci. U.S.A. 96:6937-6941, 1999) and Werner syndrome protein (Blander et al., J. Biol. Chem. 274:29463-29469, 1999; Spillare et al., Genes Dev. 13:1355-1360, 1999). ARF knockout mice show increased resistance to senescence (Kamijo et al., Cell 91:649-659, 1997) and patients carrying WRN gene mutations exhibit increased genomic instability and premature aging. A recent report demonstrates that in mice with a gain-of-function p53 mutation there is a remarkable acceleration of aging and reduced longevity (Tyner et al., Nature 415:45-53, 2002). The results disclosed herein represent a novel control mechanism of cell-cycle progression in stem cells and cancer cells involving a new nucleolar protein that can function through known tumor suppressor genes in late S and G₂. The complementary expression patterns of nucleostemin and ARF suggest a switch in the nucleolar control of cell proliferation during stem cell differentiation.

### Example 12

### Identification of Additional Nucleostemin Sequences

Two additional related nucleostemin genes were identified by sequence comparison and homology analysis. Each of these sequences contains the GTP-binding motif The first, termed NS2, is closely related to nucleostemin. The nucleic acid sequence of NS2 mRNA is set forth as Genbank Accession No. BC020354, the nucleic acids sequence of NS mRNA is set forth as Accession No. XM_{—}136118, while the protein sequence is set forth as Accession No. XP_{—}136118. These Genbank entries are incorporated herein in their entirety. In the NS2 nucleic acids sequence shown below (SEQ ID NO: 11), the coding sequence is indicated in bold.

GCTGCGCACTCCTGGACTGGCGACGTTGTGCTTCTAACAGCTCTCCGAGG
TCCCTGCCGGAAGTGTAGGAAGAAGCAGACAGATTTGAACATCTCTGTTT
CCAGCTTCTCTGATCATCATGATGAAGATTAGACACAAAAACAAAAAACC
AGGTAAAGGTTCCAAAGGCTGTAAGAAGCCTGCAAGGCAAAATGGGAAGA
AAGTAACCTCCAGACCATCATCTGCTCCCCAGATTGTTCATGGCAATGAC
CATGCCAGTCGCGAGGCCGAATTAAAGAAGAAAAGGGTCGAGGAGATGAG
GGAGAAGCAGCAAGTTGCCCGGGAGCAAGAGAGACAGAGACACAGGACCA
TGGAGAGCTATTGTCAGGATGTCCTGAAACGTCAGCAGGAATTTGAACAA
AAGGAGGAAGTTTTGCAGGAATTAAACATGTTTCCTCAGTTGGATGATGA
GGCCACAAGGAAGGCCTATTACAAGGAATTCCGGAAGGTGGTAGAGTACT
CTGATGTGATTCTGGAAGTCCTAGATGCCAGAGACCCATTGGGCTGCCGC
TGTTTCCAGATGGAGGAGACTGTCCTTCGTGCAGAAGGCAACAAGAAGCT
GGTCTTGTTCTTAAATAAGATAGATCTCGTTCCCAAGGAGATTGTGGAAA
AGTGGCTGGAATACCTTCTCAATGAACTGCCAACTGTGGCTTTCAAGGCC
AGCACCCAGCATCATCAGGTCAAAAACTTGACTCGTTGTAAAGTTCCAGT
GGACCAGGCCTCTGAGTCGCTTTTGAAAAGCAGAGCCTGCTTTGGAGCCG
AAAATCTCATGAGGGTCCTGGGGAACTATTGTCGCCTGGGGGAAGTGCGT
GGCCACATTCGTGTGGGTGTTGTAGGCCTTCCCAATGTGGGGAAGAGCAG
TCTGATCAATAGCCTGAAGCGCAGCCGTGCTTGTAGTGTGGGAGCTGTTC
CTGGTGTCACAAAATTCATGCAGGAGGTCTACCTAGACAAGTTTATCAGG
CTTCTGGATGCACCAGGCATTGTCCCAGGACCCAATTCAGAGGTGGGCAC
CATCCTGCGTAATTGCATCCATGTGCAGAAGCTGGCAGACCCTGTGACCC
CGGTGGAGACCATCCTTCAGCGCTGCAACCTGGAGGAGATTTCCAGCTAC
TATGGBTGTATCTGGATTCCAGACGACTGAGCACTTTCTGACTGCAGTGG
CCCATCGCTTGGGAAAGAAGAAGAAGGGAGGTGTATATAGTCAGGAACAG
GCTGCCAAAGCTGTGCTGGCTGACTGGGTGAGTGGGAAGATCAGCTTCTA
TACACTACCACCGCCCACTCACACTCTGCCCACCCATCTCAGTGCTGAGA
TTGTTAAGGAGATGACTGAGGTCTTTGATATAGAAGATACTGAGCACGCC
AATGAAGACACCATGGAATGCTTAGCTGTGGGAGAATCCGATGAGCTGTT
GGGTGACATGGACCCACAAGAAATGGAGGTCAGGTGGCTCCATTCTCCAC
TGGTGAAAATAGCAGATGCTATTGAAAATAGAAGCACCGTGTATAAGATT
GGAAATCTCACTGGGTATTGTACCAAACCAAACCGTAATCAGATGGGGTG
GCCTAAACGCAATGTGGACCACCATTGCCCCCAAAATAACCGTGTAGTAG
AGGTCAGTTCTGTGGACCGCCGCCCGATGTTGCAGAGGATCCTGGAGACA
GACCCACTTCAGCAAGGCCAGGCTCTGGAATCTGCCTTGAAGAATAAGAA
AAAATTGCAGAAGCGTTCAGATAAAATCGCCACTAAGTTGTCTGACTCCA
TGATGTCCATGCTTGACCTCTCTGGCAACTCCGATGACTGTGCAGGTGAC
TGAGCAGCTGACCTTCCCCCTCATACTGCAAGTACTGCTTCCCGTGC
GATGGGAGAGTCAGATGCCTTTCATTCTCT

The amino acid sequence of NS2 (SEQ ID NO: 12) is shown below:

MMKIRHKNKKPGKGSKGCKKPARQNGKKVTSRPSSAPQIVHGNDHASREA
ELKKKRVEEMREKQQVAREQERQRHRTMESYCQDVLKRQQEFEQKEEVLQ
ELNMFPQLDDEATRKAYYKEFRKVVEYSDVILEVLDARDPLGCRCFQMEE
TVLRAEGNKKLVLVLNKIDLVPKEIVEKWLEYLLNELPTVAFKASTQHHQ
VKNLTRCKVPVDQASESLLKSRACFGAENLMRVLGNYCRLGEVRGHIRVG
VVGLPNVGKSSLINSLKRSRACSVGAVPGVTKFMQEVYLDKFIRLLDAPG
IVPGPNSEVGTILRNCIHVQKLADPVTPVETILQRCNLEEISSYYGVSGF
QTTEHFLTAVAHRLGKKKKGGVYSQEQAAKAVLADWVSGKISFYTLPPPT
HTLPTHLSAEIVKEMTEVFDIEDTEHANEDTMECLAVGESDELLGDMDPQ
EMEVRWLHSPLVKIADAIENRSTVYKIGNLTGYCTKPNRNQMGWPKRNVD
HHCPQNNRVVEVSSVDRRPMLQRILETDPLQQGQALASALKNKKKLQKRS
DKIATKLSCSMMSMLDLSGNSDDCAGD

The nucleic acid sequence of NS2 mRNA is set forth as Genbank Accession No. XM_{—}131661, the nucleic acids sequence of NS mRNA is set forth as Accession No. NM145552, while the protein sequence is set forth as Accession No. NP663527 and AAH03262. These Genbank entries are incorporated herein in their entirety. In the NS3 sequence (SEQ ID NO: 13) shown below, the coding sequence is indicated in bold.

GTCGACCCACGCGTCCGCCAACGTGCTTCACACAGGACGCTTTCCCGGCT
GGGAAGTCGTGAAGTGGTTTACCTCGCGAGTAGCAGAGCGGTGTCGGTAC
TGTCGTCAGGATGGTGAAGCCCAAGTACAAAGGACGGAGCACCATCAACC
GCTCGGCGGCCAGCACCAACCCAGATCGAGTACAGGGAGCTGGCGGCCAA
AACATGAGGGATCGGGGCACAATTCGGCGCCTGAATATGTACAGGCAAAA
GGAGCGCAGGAACAGTCGTGGTAAAGTGATTAAGCCACTGCAGTATCAGT
CAACTGTGGCTTCTGGCACAGTGGCCCGAGTGGAGCCGAATATTAAATGG
TTTGGAAATACTCGTGTGATCAAGCAGGCATCATTACAAAAATTTCAAGA
GGAAATGGATAAAGTTATGAAGGATCCATACAAAGTTGTCATGAAACAAA
GCAAATTACCGATGTCTCTTCTGCACGATCGAATCCAGCCTCATAACGCA
AAAGTCCACATTCTTGATACTGAAAGCTTTGAAAGTACATTEGGCCCAAA
GTCACAGAGAAAGCGGCCAAACTTGTTTGCAAGTGATATGCAATCCCTTC
TAGAAAACGCTGAAATGTCTACTGAGAGCTATGACCAGGGCAAGGACCGT
GATTTGGTGATGGAGGACACTGGTGTAAGAAATGAAGCTCAAGAAGAGAT
ATATAAAAAAGGGCAGTCAAAAAGAATATGGGGAGAACTCTACAAGGTGA
TAGACTCATCAGATGTTGTCGTTCAAGTCCTTGACGCTAGAGATCCGATG
GGCACTCGTTCCCCCCACATCGAAGCTTACTTGAAAAAGGAAAAACCCTG
GAAACATCTCATTTTTGTACTCAATAAGTGTGACCTTGTTCCAACTTGGG
CAACCAAACGATGGGTTGCTGTGCTCTCCCAGGACTACCCAACACTGGCT
TTCCATGCGAGCCTCACCAATCCCTTTGGGAAGGGAGCATTCATTCAGCT
TCTGCGGCAGTTTGGGAAGTTGCACACAGACAAGAAACAAATCAGTGTTG
GGTTCATTGGCTATCCAAATGTAGGCAAGAGCTCTGTGATTAATACATTA
CGATCCAAGAAAGTTTGCAACGTGGCCCCCATTGCTGGAGAAACAAAGGT
CTGGCAGTATATTACCTTGATGCGTCGTATATTCCTGATTGACTGCCCTG
GTGTGGTTTACCCATCTGAGGACTCAGAGACCGACATTGTGCTCAAAGGA
GTGGTTCAAGTTGAGAAAATTAAAGCTCCTCAAGACCACATTGGTGCTGT
CCTTGAACGAGCAAAGCCAGAGTATATCAGCAAGACGTACAAGATTGAGT
CCTGGGAGAACGCGGAGGACTTTCTTGAGAAGCTAGCTCTCCGCACTGGG
AAGTTACTGAAGGGTGGAGAGCCTGACATGCTGACTGTGAGCAAGATGGT
TCTCAATGACTGGCAGAGAGGCCGAATCCCTTTCTTTGTCAAGCCGCCCA
ATGCAGAGCTACCGACCGATTCCCAGCTTCCACCATCCTCACCATTGGAA
GTTCCCACAGAAACAACCCAGAACAACCCAGAAGAAGAGACCACAGAAAC
AGAAGTTGAAAGGTCAGACTCTATCACTGAAAAGGAGCCAGAAGGAGACT
GTTCTCAGGATAGAAACTCAGAGATGCAACAGATCCTCGCACGAGTTCGC
CAGAACTTTGGCAAAATCAACGTGGGGCCTCAGTTTTCTGCGGATGACCT
GGTGCCTGTGGAGATGTCAGACTTGGAAGATCTGGAAAGCTCTGGGGAAG
AGGAAGAACAGGAGCAGGAACAGCCAGGGGAGGATGCCGAGGAAGAGCGC
TCCCCAGACACTCAGGAGGAACCAGTGGGAAACGACACCAAGGCCGTGCT
CAGAGCCCTGGATGAGAAGATTGCCAAGTACCAGAGGTTTCTAAATAAAG
CTAAAGCTAAAAAGTTCTCTGCCGTCAGAATATCCAAGGACTTAAGTGAA
AAGGTTTTTGCAAAATACAAAGAAGAGAAGAAAACATCTGCAGAAGACAG
TGATGCAGCACCCACCAAAAAGGCAAGGAAGTGGGATGCACAGATGGAAG
AAGAACCTTCAAATAAGACTCAGAGGATGCTGACGTGTAAGGAACGGAGG
AGAGCAGCACGGCAGCAACAATCCAAAAAAGTTGGTGTGCGTTACTACGA
GACACACAATGTGAAAAACAGGAACAGGAACAAAAAGAAGACGAGCGACT
CAGAGGGACAGAAACACAGACGCAACAAGTTCAGACAGAAGCAGTAACTG
CGAGAAAGCTGTTTATTAAATTATACAAAAATAAAAAAAAAAAAAAAAAG
GGCGGCCGC

The amino acid sequence of NS3 (SEQ ID NO: 14) is shown below:

MVKPKYKGRSTINRSAASTNPDRVQGAGGQNMRDRGTIRRLNMYRQKERR
NSRGKVIKPLOYQSTVASGTVARVEPNIKWFGNTRVIKQASLQKFQEEMD
KVMKDPYKVVMKQSKLPMSLLHDRIQPHNAKVHILDTESFESTFGPKSQR
KRPNLFASDMQSLLENAEMSTESYDQGKDRDLVMEDTGVRNEAQEEIYKK
GQSKRIWGELYKVIDSSDVVVQVLDARDPMGTRSPHIEAYLKKEKPWKHL
IFVLNKCDLVPTWATKRWVAVLSQDYPTLAFHASLTNPFGKGAFIQLLRQ
FGKLHTDKKQISVGFIGYPNVGKSSVINTLRSKKVCNVAPIAGETKVWQY
ITLMRRIFLIDCPGVVYPSEDSETDIVLKGVVQVEKIKAPQDHIGAVLER
AKPEYISKTYKIESWENAEDFLEKLALRTGKLLKGGEPDMLTVSKMVLND
WQRGRIPFFVKPPNAEIPTDSQLPPSSPLEVPTETTQNNPEEETTETEVE
RSDSITEKEPEGDCSQDRNSEMQQILARVRQNFGKINVGPQFSADDLVPV
EMSDLEDLESSGEEEEQEQEQPGEDQEEERSPDTQEEPVGNDTKAVLRAL
DEKIAKYQRFLNKAKAKKFSACRISKDLSEKVFAKYKEEKKTSAEVSDAA
PTKKARKWDAQMEEEPSNKTQRMLTCKERRRAARQQQSKKVGVRYYETHN
VKNRNRNKKKTSDSEGQKHRRNKFRQKQ*

The NS2 and NS3 amino acid and polypeptide sequences, or a sequence at least about 85%, at least about 90% at least about 95%, or at least about 99% homologous to a NS2 or NS3 sequence can be used in the methods disclosed herein. Both NS2 and NS3 contain a GTP binding motif.

A Northern blot analysis was performed to determine the expression of NS2 and NS3 in cultures of stem cells and in cultures of differentiated cells. As noted for human nucleostemin, the mRNA encoding NS3 was highly expressed in stem cell. In addition, the expression of NS3 was decreased in differentiated cells. However, the expression of NS2 was higher in stem cells than in differentiated cells.

Antibodies were produced that specifically bind NS2 and NS3.

It will be apparent that the precise details of the methods or compositions described may be varied or modified without departing from the spirit of the described invention. We claim all such modifications and variations that fall within the scope and spirit of the claims below.

1-22. (canceled)

23. A method for inducing differentiation or inhibiting proliferation of a cell, comprising
altering the level of a nucleostemin polypeptide comprising an amino acid sequence at least 80% identical to SEQ ID NO: 6 in the cell,
thereby inducing differentiation or inhibiting proliferation of the cell.

24. The method of claim 23, wherein altering the level of the nucleostemin polypeptide comprises increasing the level of the polypeptide.

25. The method of claim 23, wherein altering the level of the nucleostemin polypeptide comprises decreasing the level of the polypeptide.

26. The method of claim 23, wherein the nucleostemin polypeptide comprises an amino acid sequence set forth as SEQ ID NO: 6.

27. The method of claim 23, wherein the nucleostemin polypeptide comprises an amino acid sequence set forth as SEQ ID NO: 4.

28. The method of claim 23, wherein the nucleostemin polypeptide comprises an amino acid sequence set forth as SEQ ID NO: 2.

29. The method of claim 23, wherein the nucleostemin polypeptide has a sequence set forth as SEQ ID NO: 10.

30. The method of claim 23, wherein the cell is a tumor cell.

31. The method of claim 23, wherein the cell is in vitro.

32. The method of claim 23, wherein the cell is in vivo.

33. The method of claim 23, wherein altering the level of the nucleostemin polypeptide comprises increasing transcription of a nucleic acid sequence encoding the nucleostemin polypeptide.

34. The method of claim 23, wherein altering the level of the nucleostemin polypeptide comprises altering the amount of the polypeptide bound to p53.

35. The method of claim 23, wherein altering the level of the nucleostemin polypeptide comprises introducing into the cell a small inhibitory RNA that specifically binds a polynucleotide encoding the nucleostemin polypeptide.

36. The method of claim 23, wherein the cell is a stem cell.

37. A method of screening for agents that affect differentiation or proliferation of a cell; comprising
contacting p53 and a nucleostemin polypeptide comprising an amino acid sequence at least 80% identical to SEQ ID NO: 6 with an agent of interest in vitro; and
evaluating binding of p53 and the nucleostemin polypeptide; wherein a decrease in the binding of p53 and the nucleostemin polypeptide as compared to a control indicates that the agent affects differentiation or proliferation of the cell.

38. The method of claim 37, wherein the control is a standard value.

39. The method of claim 37, wherein the control is the binding of p53 and the nucleostemin polypeptide in the absence of the agent.

40. A method of inducing senescence of a cell, comprising
altering the level of a polypeptide comprising an amino acid sequence at least 80% identical to SEQ ID NO: 6, thereby inducing senescence of the cell.

41. The method of claim 40, wherein altering the level of the nucleostemin polypeptide comprises increasing the level of the polypeptide.

42. The method of claim 40, wherein altering the level of the nucleostemin polypeptide comprises decreasing the level of the polypeptide.

43. The method of claim 40, wherein the nucleostemin polypeptide comprises an amino acid sequence set forth as SEQ ID NO: 6.

44. The method of claim 40, wherein the nucleostemin polypeptide comprises an amino acid sequence set forth as SEQ ID NO: 4.

45. The method of claim 40, wherein the nucleostemin polypeptide comprises an amino acid sequence set forth as SEQ ID NO: 2.

46. The method of claim 40, wherein the nucleostemin polypeptide comprises an amino acid sequence set forth as SEQ ID NO: 10.

47. The method of claim 40, wherein the cell is a tumor cell.

48. The method of claim 40, wherein the cell is a stem cell.

49. The method of claim 40, wherein the cell is in vitro.

50. The method of claim 40, wherein the cell is in vivo.

51. A method of decreasing proliferation of a tumor cell in a subject, comprising administering to the subject a therapeutically effective amount of an agent that alters the level of a nucleostemin polypeptide comprising an amino acid sequence at least 80% identical to SEQ ID NO: 6, thereby decreasing proliferation of the tumor cell in the subject.

52. The method of claim 51, wherein the agent is a small inhibitory RNA that specifically binds a polynucleotide encoding the nucleostemin polypeptide.

53. The method of claim 51, wherein the agent is a polynucleotide encoding a nucleostemin polypeptide.

54. The method of claim 51, wherein the agent is a p53.

55. A method for inducing differentiation, inducing senescence, or inhibiting proliferation of a cell, comprising
altering the level of a nucleostemin polypeptide comprising an amino acid sequence set forth as SEQ ID NO: 10 in the cell,
thereby inducing differentiation, inducing senescence or inhibiting proliferation of the cell.

56. The method of claim 55, wherein the cell is a tumor cell.

57. The method of claim 55, wherein the cell is in vitro.

58. The method of claim 55, wherein the cell is in vivo.

59. The method of claim 55, wherein altering the level of the nucleostemin polypeptide comprises increasing transcription of a nucleic acid sequence encoding the nucleostemin polypeptide.

60. The method of claim 55, wherein altering the level of the nucleostemin polypeptide comprises altering the amount of the polypeptide bound to p53.

61. The method of claim 55, wherein altering the level of the nucleostemin polypeptide comprises introducing into the cell a small inhibitory RNA that specifically binds a polynucleotide encoding the nucleostemin polypeptide.

62. The method of claim 55, wherein the cell is a stem cell.

63. An antibody that specifically binds the polypeptide of claim 1.
